(19) United States
(12) Patent Application Publication
(10) Pub. No.: US 2019/0265164 A1
(43) Pub. Date: Aug. 29, 2019

(54) A METHOD FOR DETERMINING THE LEVEL OF A POLYCYCLIC COMPOUND OF INTEREST PRESENT ON THE SURFACE OF A TOBACCO LEAF

(71) Applicant: BRITISH AMERICAN TOBACCO (INVESTMENTS) LIMITED, London (GB)

(72) Inventors: Jana Ticha, London (GB); Christopher Wright, London (GB)

(21) Appl. No.: 16/345,817

(22) PCT Filed: Nov. 1, 2017
(86) PCT No.: PCT/GB2017/053282

§ 371 Date: Apr. 29, 2019

(30) Foreign Application Priority Data
Nov. 3, 2016 (GB) 1618560.5

(51) Int. Cl. G01N 21/64; A24B 3/18; G01N 33/00

(52) U.S. Cl. CPC G01N 21/64; G01N 2021/6421; G01N 33/0098; A24B 3/18

(57) ABSTRACT

There is provided a method for determining the level of a polycyclic compound of interest present on the surface of a tobacco leaf, the method comprising the steps of: (a) washing the surface of the tobacco leaf with a solvent, the solvent comprising at least a non-polar solvent, such that no greater than about 10 wt. % of the polycyclic compound of interest present on the surface of the tobacco leaf is removed from the surface of the tobacco leaf; (b) collecting the solvent from step (a); and (c) subjecting the collected solvent to fluorescence spectroscopy to determine the level of the polycyclic compound of interest.

### FIELD OF THE INVENTION

The present disclosure relates to a method for determining the level of a polycyclic compound of interest in tobacco, and a device for determining the same.

### BACKGROUND TO THE INVENTION

Polycyclic compounds, such as polycyclic aromatic hydrocarbons (PAHs), are known carcinogens that do not occur naturally in tobacco, but may be transferred to tobacco during curing (for example, in flue cured tobacco) and/or transport, and transferred to cigarette smoke from tobacco.

It is therefore of importance to determine the level of PAHs and other toxicants in tobacco leaves. The level of PAHs in cured tobacco is one criterion used for assessing tobacco leaf quality. Gas chromatography-mass spectrometry (GC/MS) is an industry standard test for determining the level of PAHs in the tobacco leaf. However, GC/MS is a complex process, resource intensive, and thus relatively expensive. Furthermore, GC/MS can only be conducted in a laboratory, which may increase the length of time taken for testing as tobacco samples must first be shipped from the field or site of use (for instance) to a laboratory for testing.

In some embodiments, the present invention seeks to provide an improved method and an improved device for determining the level of a polycyclic compound of interest on the surface of tobacco leaves.

### SUMMARY OF THE INVENTION

Aspects of the invention are defined in the accompanying claims.

In accordance with some embodiments described herein, there is provided a method for determining the level of a polycyclic compound of interest present on the surface of a tobacco leaf, the method comprising the steps of:

(a) washing the surface of the tobacco leaf with a solvent, the solvent comprising at least a non-polar solvent, such that no greater than about 10 wt % of the polycyclic compound of interest present on the surface of the tobacco leaf is removed from the surface of the tobacco leaf;
(b) collecting the solvent from step (a); and
(c) subjecting the collected solvent to fluorescence spectroscopy to determine the level of the polycyclic compound of interest.

In accordance with some embodiments described herein, there is provided a device for determining the level of a polycyclic compound of interest present in a solvent, the device comprising:

(a) a receptacle for the solvent containing the polycyclic compound of interest;
(b) an excitation source for initiating fluorescence of the polycyclic compound of interest;
(c) a sensor for determining fluorescence; and
(d) a processor operable to
(i) identify at least a first peak and a second peak in the emission spectrum of the collected solvent, the first peak corresponding to a first specific emission wavelength of the polycyclic compound of interest and the second peak corresponding to a second specific emission wavelength of the polycyclic compound of interest;
(ii) compare the intensities of the at least two peaks relative to each other so as to obtain a fluorescence ratio; and
(iii) compare the fluorescence ratio to pre-determined fluorescence ratios in order to determine the level of the polycyclic compound of interest.

We have surprisingly found that the method described herein may be carried out to successfully determine the level of a polycyclic compound of interest on the surface of a tobacco leaf in a relatively short timeframe. In other words, it has been found that the method described herein may be carried out relatively rapidly in order to provide the results of the testing on a shorter timescale than previously known methods. The method described herein has also been found to be able to be carried out on a small-scale, and for example using a portable device, such as a hand-held device, such that the level of polycyclic compounds on the surface of tobacco leaves may be carried out in situ (i.e. in the field, at the curing barns, in Green Leaf Threshing (GLT) environment, at quality control laboratories, at points of purchase, at leaf sales, in quality control across supply chain, or at smoking article production sites for example).

For ease of reference, these and further aspects of the present invention are now discussed under appropriate section headings. However, the teachings under each section are not necessarily limited to each particular section.

### BRIEF DESCRIPTION OF THE FIGURES

Embodiments of the present invention are described, by way of example only, with reference to the accompanying drawings in which:

FIG. 1 shows the absorbance spectrum of benzo[a]pyrene.

FIG. 2 shows the excitation-emission matrix for benzo[a]pyrene dissolved in methanol.

FIG. 3 shows the excitation-emission matrix for benzo[a]pyrene dissolved in hexane.

FIG. 4 shows a comparison of the fluorescence emission spectra in methanol and hexane extracted from the excitation-emission matrices at a fixed excitation wavelength of 255 nm.

FIG. 5 shows the excitation-emission matrices for direct leaf analysis with four repeats of different leaf areas of a low benzo[a]pyrene level tobacco leaf sample.

FIG. 6 shows the excitation-emission matrices for direct leaf analysis with three repeats of different leaf areas of a medium benzo[a]pyrene level tobacco leaf sample.

FIG. 7 shows the excitation-emission matrices for direct leaf analysis with four repeats of different leaf areas of a high benzo[a]pyrene level tobacco leaf sample.

FIG. 8 shows the excitation-emission matrices for blank (top left), 30 s (top right), 60 s (bottom left) and 180 s (bottom right) methanol leaf wash samples for high level benzo[a]pyrene tobacco leaves.

FIG. 9a shows the excitation-emission matrix for the tobacco leaf sample having a high level of benzo[a]pyrene that has been washed in hexane for 300 seconds.

FIG. 9b shows the excitation-emission matrix for the tobacco leaf sample having a high level of benzo[a]pyrene that has been washed in methanol for 60 seconds.

FIG. 10 shows the blank excitation-emission matrices for 30 s (top left), 60 s (top right), 180 s (bottom left) and 300 s (bottom right) wash in hexane solvent only.

FIG. 11 shows the excitation-emission matrices for the tobacco leaf samples having a low level of benzo[a]pyrene that have been washed in hexane for periods of time of 30 s (top row), 60 s (second row), 180 s (third row) and 300 s (bottom row).

FIG. 12 shows the excitation-emission matrices for the tobacco leaf samples having a high level of benzo[a]pyrene that have been washed in hexane for periods of time of 30 s (top row), 60 s (second row), 180 s (third row) and 300 s (bottom row).

FIG. 13 shows the emission spectra for the hexane leaf wash samples for low levels of benzo[a]pyrene extracted from the excitation-emission matrices at a fixed excitation wavelength of 255 nm.

FIG. 14 shows the emission spectra for the hexane leaf wash samples for high levels of benzo[a]pyrene extracted from the excitation-emission matrices at a fixed excitation wavelength of 255 nm.

FIG. 15 shows the average fluorescence for the hexane leaf wash samples at an emission peak of 330 nm with a 10 nm (±5 nm) bandpass at varying concentrations of benzo[a]pyrene.

FIG. 16 shows the average fluorescence for the hexane leaf wash samples at an emission peak of 400 nm with a 10 nm (±5 nm) bandpass at varying concentrations of benzo[a]pyrene.

FIG. 17 shows the average fluorescence ratio (400/330 nm) for the hexane leaf wash samples at varying concentrations of benzo[a]pyrene.

FIG. 18 shows the excitation-emission matrices for 2 calibration tobacco leaf samples washed in hexane for a time period of 180 s, wherein the samples each have low levels of benzo[a]pyrene.

FIG. 19 shows the excitation-emission matrices for 2 calibration tobacco leaf samples washed in hexane for a time period of 180 s, wherein the samples each have medium levels of benzo[a]pyrene.

FIG. 20 shows the excitation-emission matrices for 2 calibration tobacco leaf samples washed in hexane for a time period of 180 s, wherein the samples each have high levels of benzo[a]pyrene.

FIG. 21 shows the emission spectra for the 6 calibration hexane leaf wash samples extracted from the excitation-emission matrices at a fixed excitation wavelength of 255 nm.

FIG. 22 shows the average fluorescence ratio (400/330 nm) for the 6 calibration hexane leaf wash samples at varying concentrations of benzo[a]pyrene.

FIG. 23 shows an augmented plot of the average fluorescence ratio (400/330 nm) for the 6 calibration hexane leaf wash samples at varying concentrations of benzo[a]pyrene, together with the 2 hexane leaf wash samples of Example 1.

FIG. 24 shows the fluorescence response for benzo[a]pyrene wavelengths comprising data points from hexane leaf wash content for low, medium and high benzo[a]pyrene content.

FIG. 25 shows the background fluorescence response comprising data points from hexane leaf wash content for low, medium and high benzo[a]pyrene content.

FIG. 26 shows the fluorescence response for chlorophyll wavelengths comprising data points from hexane leaf wash content for low, medium and high benzo[a]pyrene content.

FIG. 27a shows the excitation-emission matrices for blind tobacco leaf samples 1 to 6.

FIG. 27b shows the excitation-emission matrices for blind tobacco leaf samples 7 to 12.

FIG. 27c shows the excitation-emission matrices for blind tobacco leaf samples 13 to 18.

FIG. 28 shows the average fluorescence ratio (400/330 nm) for each blind tobacco leaf samples at varying concentrations of benzo[a]pyrene.

FIG. 29 shows the fluorescence ratio (400/330 nm) for each blind tobacco leaf sample together with the 6 calibration samples at varying concentrations of benzo[a]pyrene.

While the invention is susceptible to various modifications and alternative forms, specific embodiments are shown by way of example in the drawings and are herein described in detail. It should be understood, however, that the drawings and detailed description of the specific embodiments are not intended to limit the invention to the particular forms disclosed. On the contrary, the invention covers all modifications, equivalents and alternatives falling within the scope of the present invention as defined by the appended claims.

### DETAILED DESCRIPTION

As described above, the present disclosure relates to a method for determining the level of a polycyclic compound of interest present on the surface of a tobacco leaf, the method comprising the steps of:

(a) washing the surface of the tobacco leaf with a solvent, the solvent comprising at least a non-polar solvent, such that no greater than about 10 wt. % of the polycyclic compound of interest present on the surface of the tobacco leaf is removed from the surface of the tobacco leaf;
(b) collecting the solvent from step (a); and
(c) subjecting the collected solvent to fluorescence spectroscopy to determine the level of the polycyclic compound of interest.

In some embodiments, the polycyclic compound of interest is a polycyclic compound wherein the cyclic ring systems are linked by tethering, fusing, via a single atom, or a combination thereof. In some embodiments, the polycyclic compound of interest comprises a number of fused rings. In some embodiments, the polycyclic compound of interest comprises from 2 to 10 fused rings. In some embodiments, the polycyclic compound of interest comprises from 2 to 7 fused rings. In some embodiments, the polycyclic compound of interest comprises from 3 to 6 fused rings. In some embodiments, the polycyclic compound of interest comprises from 4 to 6 fused rings. In some embodiments, the polycyclic compound of interest comprises 5 fused rings.

In some embodiments, the polycyclic compound of interest is an aromatic polycyclic compound. As used herein, the term “aromatic polycyclic compound” refers to a polycyclic compound composed of multiple aromatic rings. In some embodiments, the polycyclic compound of interest is a polycyclic aromatic hydrocarbon (PAH). As used herein, the term “polycyclic aromatic hydrocarbon” refers to an aromatic polycyclic compound which comprises only carbon and hydrogen atoms. In some embodiments, the polycyclic compound of interest is a heteroaromatic polycyclic compound. As used herein, the term “heteroaromatic polycyclic compound” refers to a polycyclic compound composed of multiple aromatic rings, wherein at least one of the aromatic ring systems comprises at least one heteroatom, such as nitrogen, sulphur, or oxygen.

In some embodiments, the polycyclic compound of interest is a polynuclear aromatic hydrocarbon (PNA). As used herein, the term “polynuclear aromatic hydrocarbon” refers to a PAH wherein the aromatic rings are fused together.

In some embodiments, the polycyclic compound of interest is a PAH (preferably a PNA) comprising from 2 to 10 hydrocarbon rings. In some embodiments, the polycyclic compound of interest is a PAH (preferably a PNA) comprising from 2 to 7 hydrocarbon rings. In some embodiments, the polycyclic compound of interest is a PAH (preferably a PNA) comprising from 3 to 6 hydrocarbon rings. In some embodiments, the polycyclic compound of interest is a PAH (preferably a PNA) comprising from 4 to 6 hydrocarbon rings. In some embodiments, the polycyclic compound of interest is a PAH (preferably a PNA) comprising 5 hydrocarbon rings.

In some embodiments, the polycyclic compound of interest is a PAH selected from the group consisting of a naphthalene, an anthracene, a phenanthrene, an acenaphthylene, an acenaphthene, a fluorene, a tetracene, a fluoranthene, a chrysene, a triphenylene, a perylene, a pyrene, a methylcholanthrene, a pentacene, a corannulene, a coronene, and an ovalene. In some embodiments, the polycyclic compound of interest is a PAH selected from benz[j]aceanthrylene, benzo[b]furan, chrysene, cyclopenta[c,d]pyrene, dibenzo[a,h]pyrene, dibenzo[a,l]pyrene, naphthalene, benz[a]anthracene, 5-methylchrysene, benzo[j]fluoranthene, benzo[b]fluoranthene, benzo[k]fluoranthene, benzo[c]phenanthrene, benzo[a]pyrene, benzo[g,h,i]perylene, methylcholanthrene, dibenz[a,h]anthracene, indeno[1,2,3-cd]pyrene, dibenzo[a,e]pyrene, and dibenzo[a,i]pyrene.

In some embodiments, the polycyclic compound of interest is a pyrene. In some embodiments, the polycyclic compound of interest is a benzopyrene. In some embodiments, the polycyclic compound of interest is a benzopyrene selected from benzo[a]pyrene (otherwise known as benzo(alpha)pyrene or BaP) and benzo[e]pyrene. In some embodiments, the polycyclic compound of interest is benzo[a]pyrene (BaP).

In some embodiments, the tobacco leaf is whole or cut tobacco leaf. In some embodiments, the tobacco leaf is whole. In some embodiments, the tobacco leaf is cut.

As described herein, the method comprises the step (a) of washing the surface of the tobacco leaf with a solvent, the solvent comprising at least a non-polar solvent, such that no greater than about 10 wt. % of the polycyclic compound of interest present on the surface of the tobacco leaf is removed from the surface of the tobacco leaf. As described herein, the solvent used in the method of the present invention comprises at least a non-polar solvent. In some embodiments, the solvent used in the method of the present invention further comprises a polar solvent, such as a polar protic solvent or a polar aprotic solvent.

As used herein, the term “polar solvent” refers to any solvent having a dielectric constant of greater than or equal to 15. As used herein, the term “non-polar solvent” refers to any solvent having a dielectric constant of less than 15. As used herein, the term “polar protic solvent” refers to any polar solvent that solvates negatively charged ions. As used herein, the term “polar aprotic solvent” refers to any polar solvent that solvates positively charged ions.

In some embodiments, the solvent used in the method of the present invention comprises an organic-based solvent. In some embodiments, the solvent used in the method of the present invention comprises a solvent selected from the group consisting of alkanes, cycloalkanes, haloalkanes, alkenes, alkynes, arenes, alcohols, aldehydes, ketones, carboxylic acids, esters, ethers, amines, amides, nitriles, sulphides, sulphoxides, water and mixtures thereof. In some embodiments, the solvent used in the method of the present invention comprises a solvent selected from the group consisting of alkanes, cycloalkanes, haloalkanes, arenes, ethers, amines and mixtures thereof. In some embodiments, the solvent used in the method of the present invention comprises a solvent selected from methanol, toluene, hexane, and mixtures thereof. In some embodiments, the solvent used in the method of the present invention comprises at least one non-polar solvent and methanol. In some embodiments, the solvent used in the method of the present invention is a mixture of methanol and toluene. In some embodiments, the solvent used in the method of the present invention is hexane.

In some embodiments, the solvent used in the method of the present invention comprises at least a non-polar solvent selected from the group consisting of pentane, hexane, heptane, cyclopentane, cyclohexane, cycloheptane, chloroform (CHCl₃), benzene, toluene, diethyl ether, 1,4-dioxane, isooctane, decane, and combinations thereof. In some embodiments, the solvent used in the method of the present invention comprises hexane, cyclohexane, or a mixture thereof. In some embodiments, the solvent used in the method of the present invention is hexane.

As described herein, the method comprises the step (a) of washing the surface of the tobacco leaf with a solvent, such that no greater than about 10 wt. % of the polycyclic compound of interest present on the surface of the tobacco leaf is removed from the surface of the tobacco leaf. In some embodiments, the method comprises the step (a) of washing the surface of the tobacco leaf with a solvent, such that from about 0.01 to about 10 wt. % of the polycyclic compound of interest present on the surface of the tobacco leaf is removed from the surface of the tobacco leaf. In some embodiments, the method comprises the step (a) of washing the surface of the tobacco leaf with a solvent, such that from about 0.1 to about 10 wt. % of the polycyclic compound of interest present on the surface of the tobacco leaf is removed from the surface of the tobacco leaf. In some embodiments, the method comprises the step (a) of washing the surface of the tobacco leaf with a solvent, such that from about 1 to about 10 wt. % of the polycyclic compound of interest present on the surface of the tobacco leaf is removed from the surface of the tobacco leaf. In some embodiments, the method comprises the step (a) of washing the surface of the tobacco leaf with a solvent, such that from about 1 to about 9 wt. % of the polycyclic compound of interest present on the surface of the tobacco leaf is removed from the surface of the tobacco leaf, such as from about 2 to about 8 wt. % of the polycyclic compound of interest present on the surface of the tobacco leaf is removed from the surface of the tobacco leaf, such as from about 3 to about 7 wt. % of the polycyclic compound of interest present on the surface of the tobacco leaf is removed from the surface of the tobacco leaf, such as from about 4 to about 6 wt. % of the polycyclic compound of interest present on the surface of the tobacco leaf is removed from the surface of the tobacco leaf.

It has been found by the present inventors that the removal of no greater than about 10 wt. % of the polycyclic compound of interest from the surface of the tobacco leaf reduces the extraction of other substances from within the leaf (such as coumarin, for example) that have very intense fluorescence spectra, and which could obscure the peaks of interest thus adversely affecting the determination of the level of a polycyclic compound of interest present on the surface of a tobacco leaf. As the skilled person will appreciate, the nature of polycyclic compound contamination of tobacco leaves, which typically occurs as a result of fugitive emissions during barn curing of tobacco, means that the polycyclic compounds are largely present on the surface of the leaf, rather than within the leaf itself. The removal of the polycyclic compound from the surface of the leaf may therefore be achieved in a controlled manner such that no greater than about 10 wt. % of the polycyclic compound of interest present on the surface of the tobacco leaf is removed from the surface of the tobacco leaf.

As used herein, the term “wt. % of the polycyclic compound of interest” refers to the percentage amount of the polycyclic compound of interest by weight of the total amount of the polycyclic compound of interest on the surface of the tobacco leaf.

In some embodiments, the method comprises the step (a) of washing the surface of the tobacco leaf (whole or cut) with a solvent, such that no greater than about 5 wt. % of the polycyclic compound of interest present on the surface of the tobacco leaf is removed from the surface of the tobacco leaf. In some embodiments, the method comprises the step (a) of washing the surface of the tobacco leaf with a solvent, such that from about 0.01 to about 5 wt. % of the polycyclic compound of interest present on the surface of the tobacco leaf is removed from the surface of the tobacco leaf. In some embodiments, the method comprises the step (a) of washing the surface of the tobacco leaf with a solvent, such that from about 0.1 to about 5 wt. % of the polycyclic compound of interest present on the surface of the tobacco leaf is removed from the surface of the tobacco leaf. In some embodiments, the method comprises the step (a) of washing the surface of the tobacco leaf with a solvent, such that from about 1 to about 5 wt. % of the polycyclic compound of interest present on the surface of the tobacco leaf is removed from the surface of the tobacco leaf. It has been found by the present inventors that the solvent wash may remove other substances from the surface of the tobacco leaf, and that the removal of no greater than 5 wt. % polycyclic compound means that the signal for the co-extracts may not overwhelm the signal for the polycyclic compound of interest.

In some embodiments, the amount of solvent used to wash the surface of the tobacco leaf is from about 10 to about 50 ml of solvent per 1 g of tobacco leaf, such as from about 15 to about 45 ml of solvent per 1 g of tobacco leaf, such as from about 20 to about 40 ml of solvent per 1 g of tobacco leaf, such as from about 20 to about 35 ml of solvent per 1 g of tobacco leaf, such as from about 20 to about 30 ml of solvent per 1 g of tobacco leaf, such as approximately 25 ml of solvent per 1 g of tobacco leaf.

In some embodiments, the tobacco leaf is washed with solvent for a period of less than 10 minutes in step (a) of the method according to the present invention, such as for a period of less than about 8 minutes, such as for a period of no greater than about 5 minutes. In some embodiments, the tobacco leaf is washed with solvent for a period of at least about 0.5 minutes in step (a) of the method according to the present invention, such as for a period of at least about 1 minute, such as for a period of at least about 1.5 minutes, such as for a period of at least about 2 minutes. In some embodiments, the tobacco leaf is washed with solvent for a period of from about 0.5 minutes to about 10 minutes in step (a) of the method according to the present invention, such as for a period of from about 1 minute to about 7 minutes, such as for a period of from about 2 minutes to about 5 minutes. In some embodiments, the tobacco leaf is washed with solvent for a period of from about 0.5 minutes to about 5 minutes.

In some embodiments, the tobacco leaf is washed with solvent in step (a) of the method according to the present invention at a temperature of about 10 to about 30° C., such as at a temperature of about 15 to about 25° C., such as at a temperature of about 20 to about 25° C.

In some embodiments, the tobacco leaf is washed with solvent in step (a) of the method according to the present invention at a pressure of from about 50 to about 150 kPa. In some embodiments, the tobacco leaf is washed with solvent in step (a) of the method according to the present invention at atmospheric pressure.

As described herein, the method comprises the step (c) of subjecting the collected solvent to fluorescence spectroscopy to determine the level of the polycyclic compound of interest. In some embodiments, the collected solvent subject to fluorescence spectroscopy contains the polycyclic compound of interest in an amount of from about 5 pg/mL to about 500 pg/mL, such as from about 10 pg/mL to about 500 pg/mL, such as from about 20 pg/mL to about 500 pg/mL, such as from about 30 pg/mL to about 500 pg/mL, such as from about 40 pg/mL to about 500 pg/mL, such as from about 50 pg/mL to about 500 pg/mL.

In some embodiments, the collected solvent subject to fluorescence spectroscopy contains the polycyclic compound of interest in an amount of from about 1 ng per g of tobacco leaf to about 500 ng per g of tobacco leaf, such as from about 5 ng per g of tobacco leaf to about 500 ng per g of tobacco leaf, such as from about 10 ng per g of tobacco leaf to about 500 ng per g of tobacco leaf.

As the skilled person will appreciate, when some dissolved aromatic compounds absorb UV light, they may re-emit a fraction of this energy as fluorescence at longer wavelengths. The fluorescence spectroscopy to which the collected solvent is subjected thus involves the excitation of molecules in the sample by absorption of incident UV radiation, and the subsequent emission (fluorescence) of the molecules as they return to their ground state configurations, where the intensity of the fluorescence at each emission wavelength is measured by a suitable detector. As the skilled person will appreciate, fluorescence intensity is directly proportional to the concentration of the sample species.

Typically, in fluorescent spectroscopy of polycyclic compounds, incident UV radiation may have a wavelength of from about 200 to about 500 nm. In embodiments of the present invention, the incident UV radiation may be provided by an excitation source, such as an incandescent tungsten-filament lamp, incandescent tungsten-halogen lamp, a discharge lamp containing xenon gas and/or mercury vapour, a laser or a UV light-emitting-diode (LED) light source.

In some embodiments, the fluorescence of the collected solvent subject to fluorescence spectroscopy is detected using a detector selected from photomultiplier (PMT) detectors, thermopiles, photocells, photodiodes and charge-coupled device (CCD) detectors. In some embodiments, the fluorescence of the collected solvent subject to fluorescence spectroscopy is detected using a PMT detector.

In some embodiments, the fluorescence spectroscopy is carried out using an apparatus capable of simultaneously measuring both absorbance spectra and emission spectra to provide excitation-emission matrices (EEMs) for the collected solvent to determine the level of the polycyclic compound of interest.

In some embodiments, the fluorescence spectroscopy comprises identifying at least a first peak and a second peak in the emission spectrum of the collected solvent, the first peak corresponding to a first specific emission wavelength of the polycyclic compound of interest and the second peak corresponding to a second specific emission wavelength of the polycyclic compound of interest.

The emission spectrum of the collected solvent constitutes the fluorescence (or emission) signal produced by the collected solvent at a certain, predetermined excitation wavelength of the incident radiation. As described herein, the first peak corresponds to a first specific emission wavelength of the polycyclic compound of interest that is discreet from the matrix background. As used herein, the matrix background refers to wavelengths in the emission spectrum of the collected solvent that is not emission produced by the polycyclic compound of interest. In some embodiments, the first peak is selected such that it constitutes an emission maximum in the emission spectrum of the collected solvent, where said emission maximum is produced by the polycyclic compound of interest.

In some embodiments, the emission spectrum is obtained at an excitation wavelength in the range of from 200 to 500 nm, such as from 200 to 400 nm, such as from 200 to 300 nm. In some embodiments, the emission spectrum is obtained at an excitation wavelength in the range of from 220 to 280 nm. In some embodiments, the emission spectrum is obtained at an excitation wavelength of 280 nm. In some preferred embodiments, the emission spectrum is obtained at an excitation wavelength of 255 nm.

In some embodiments (such as where the excitation wavelength is 255 nm), the first peak is emission with a wavelength in the range of from 300 to 370 nm, such as in the range of from 310 to 370 nm. In some embodiments (such as where the excitation wavelength is 255 nm), the first peak is emission with a wavelength in the range of from 310 to 360 nm. In some embodiments (such as where the excitation wavelength is 255 nm), the first peak is emission with a wavelength in the range of from 320 to 350 nm. In some embodiments (such as where the excitation wavelength is 255 nm), the first peak is emission with a wavelength in the range of from 320 to 340 nm. In some embodiments (such as where the excitation wavelength is 255 nm), the first peak is emission with a wavelength in the range of from 325 to 335 nm. In some embodiments (such as where the excitation wavelength is 255 nm), the first peak is emission with a wavelength of approximately 330 nm. As used herein, the term “approximately 330 nm” refers to an emission peak at 330 nm, including a bandpass of ±5 nm for the emission peak (i.e. 330 nm±5 nm).

In some embodiments, in the emission spectrum of the collected solvent produced using an excitation wavelength of 255 nm, the first peak is emission with a wavelength of from 320 to 340 nm. In some embodiments, in the emission spectrum of the collected solvent produced using an excitation wavelength of 255 nm, the first peak is emission with a wavelength of approximately 330 nm.

In some embodiments, in the emission spectrum of the collected solvent produced using an excitation wavelength of 255 nm, e.g. where the solvent is a non-polar solvent, e.g. hexane, the first peak is emission with a wavelength in the range of from 325 to 335 nm, such as approximately 330 nm.

In some embodiments (such as where the excitation wavelength is 255 nm), the second peak is emission with a wavelength in the range of from 385 to 425 nm, e.g. in the range of from 390 to 420 nm, such as in the range of from 390 to 410 nm.

In some embodiments (such as where the excitation wavelength is 255 nm), the second peak is emission with a wavelength in the range of from 395 to 405 nm, such as approximately 400 nm. As used herein, the term “approximately 400 nm” refers to an emission peak at 400 nm, including a bandpass of ±5 nm for the emission peak (i.e. 400 nm±5 nm).

In some embodiments, in the emission spectrum of the collected solvent produced using an excitation wavelength of 255 nm, e.g. where the solvent is a non-polar solvent, e.g. hexane, the second peak is emission with a wavelength in the range of from 395 to 405 nm, such as approximately 400 nm.

In some embodiments, the fluorescence spectroscopy comprises identifying at least a first peak and a second peak in the emission spectra of the collected solvent, the first peak corresponding to a first specific emission wavelength of the polycyclic compound of interest and the second peak corresponding to a second specific emission wavelength of the polycyclic compound of interest, wherein the fluorescence spectroscopy further comprises the step of comparing the intensities of the first peak and the second peak relative to each other so as to obtain a fluorescence ratio. As used herein, the term “fluorescence ratio” refers to a ratio of the fluorescence intensity of a second peak in the emission spectrum of the collected solvent to the fluorescence intensity of a first peak in the emission spectrum, wherein the first peak corresponds to a first specific emission wavelength of the polycyclic compound of interest and the second peak corresponds to a second specific emission wavelength of the polycyclic compound of interest.

In some embodiments, the fluorescence spectroscopy comprises identifying two or more peaks in the emission spectrum of the collected solvent, wherein at least two of the peaks correspond to specific emission wavelengths of the polycyclic compound of interest, and comparing the intensities of the peaks corresponding to specific emission wavelengths of the polycyclic compound of interest so as to obtain a fluorescence ratio. In some embodiments, the fluorescence spectroscopy comprises identifying three peaks in the emission spectrum of the collected solvent, wherein one of the three peaks corresponds to a specific emission wavelength of the polycyclic compound of interest, and comparing the intensities of the peak corresponding to a specific emission wavelength of the polycyclic compound of interest relative to the intensities of the two remaining peaks so as to obtain a fluorescence ratio.

In some embodiments, the fluorescence spectroscopy comprises identifying three or more peaks in the emission spectrum of the collected solvent, wherein at least one of the three or more peaks corresponds to a specific emission wavelength of the polycyclic compound of interest, and comparing the intensities of the at least one peak corresponding to a specific emission wavelength of the polycyclic compound of interest relative to the intensities of the two or more remaining peaks so as to obtain a fluorescence ratio. In some embodiments, the fluorescence spectroscopy comprises identifying three peaks in the emission spectrum of the collected solvent, wherein one of the three peaks corresponds to a specific emission wavelength of the polycyclic compound of interest, and comparing the intensities of the peak corresponding to a specific emission wavelength of the polycyclic compound of interest relative to the intensities of the two remaining peaks so as to obtain a fluorescence ratio.

In some embodiments, the method described herein further comprises the step of comparing the fluorescence ratio to pre-determined (or calibration) fluorescence ratios in order to determine the level of the polycyclic compound of interest on the surface of the tobacco leaf. The pre-determined fluorescence ratios are calculated for at least two reference samples which have a known level of the polycyclic compound of interest, wherein at least one sample has a low level of the polycyclic compound of interest and at least one sample has a high level of the polycyclic compound of interest. A low level of the polycyclic compound of interest is typically considered to be an amount of less than 10 ng g⁻¹ of the polycyclic compound of interest on the surface of the tobacco leaf, while a high level of the polycyclic compound of interest is typically considered to be an amount of greater than 300 ng g⁻¹ of the polycyclic compound of interest on the surface of the tobacco leaf. A comparison of the fluorescence ratio obtained using the method of the present invention to such fluorescence ratios calculated for samples having a known level of the polycyclic compound of interest therefore facilitates the determination of the level of the polycyclic compound of interest washed from the surface of the tobacco leaf.

The present invention also provides a device for determining the level of a polycyclic compound of interest present in a solvent, the device comprising:

(a) a receptacle for the solvent containing the polycyclic compound of interest;
(b) an excitation source for initiating fluorescence of the polycyclic compound of interest;
(c) a sensor for determining fluorescence; and
(d) a processor operable to
(i) identify at least a first peak and a second peak in the emission spectrum of the collected solvent, the first peak corresponding to a first specific emission wavelength for the polycyclic compound of interest and the second peak corresponding to a second specific emission wavelength of the polycyclic compound of interest;
(ii) compare the intensities of the two peaks relative to each other so as to obtain a fluorescence ratio; and
(iii) compare the fluorescence ratio to pre-determined fluorescence ratios in order to determine the level of the polycyclic compound of interest.

In some embodiments, the device is portable. As used herein, the term “portable” means able to be carried or moved with ease. In some embodiments, the device may be of a size such that it can be held in the hand of a user without discomfort during use. In some embodiments, the device is a portable, hand-held device.

In some embodiments, the excitation source is a UV radiation source selected from the group consisting of an incandescent tungsten-filament lamp, incandescent tungsten-halogen lamp, a discharge lamp containing xenon gas and/or mercury vapour, a laser or a UV LED light source. In some embodiments, the excitation source is a UV LED light source, which is a monochromatic light source.

In some embodiments, the sensor for determining fluorescence is selected from photomultiplier (PMT) detectors, thermopiles, photocells, photodiodes and charge-coupled device (CCD) detectors. In some embodiments, the sensor for determining fluorescence is a PMT detector.

In some embodiments, the device is a UviLux Sensor as produced by Chelsea Technologies Group Ltd (CTG).

In some embodiments, the processor is operable to
(i) identify two or more peaks in the emission spectrum of the collected solvent, wherein at least two of the peaks corresponds to specific emission wavelengths of the polycyclic compound of interest;
(ii) compare the intensities of the peaks corresponding to specific emission wavelengths of the polycyclic compound of interest so as to obtain a fluorescence ratio; and
(iii) compare the fluorescence ratio to pre-determined fluorescence ratios in order to determine the level of the polycyclic compound of interest.

In some embodiments, the processor is operable to
(i) identify three peaks in the emission spectrum of the collected solvent, wherein one of the three peaks corresponds to a specific emission wavelength of the polycyclic compound of interest;
(ii) compare the intensities of the peak corresponding to a specific emission wavelength of the polycyclic compound of interest relative to the intensities of the two remaining peaks so as to obtain a fluorescence ratio; and
(iii) compare the fluorescence ratio to pre-determined fluorescence ratios in order to determine the level of the polycyclic compound of interest.

The invention will now be described with reference to the following non-limiting examples.

### EXAMPLES

### Example 1

Three different sample preparations were assessed for their ability to determine the level of benzo[a]pyrene (BaP) on the surface of a tobacco leaf.

### Samples Tested

Cured tobacco leaf samples were selected as tobacco leaf samples having known contents of BaP, which are defined as either low, medium or high levels of BaP, as shown in Table 1.

TABLE 1
cured tobacco leaf samples having known BaP content

| Sample | | BaP content | BaP content | BaP content |
|---|---|---|---|---|
| Number | Weight (g) | (ng g⁻¹) | (ppb g⁻¹) | (nM g⁻¹) |
| 259 | 100 g | 3.85 | 0.154 | 0.610 |
| 261 | 100 g | 256.91 | 10.276 | 40.279 |
| 263 | 50 g | 509.64 | 20.386 | 80.796 |

Sample number 259 was selected as having a low level of BaP, sample number 261 was selected as having a medium level of BaP, and sample number 263 was selected as having a high level of BaP.

Each of these samples were analysed as set out in points (1) to (3) below. It is noted that the analyses conducted in points (1) and (2) below can be regarded as comparative examples.
(1) Tobacco leaf samples with known low, medium and high BaP content were investigated by measuring excitation-emission matrices directly on the tobacco leaves. For each BaP content leaf, three to four repeats of the leaf fluorescence in different parts of the leaf were acquired to assess whether the variation within leaves having the same BaP content was significant compared to the differences between leaves having either low, medium and high BaP content.
(2) Tobacco leaf samples with known low, medium and high BaP content were washed with methanol at ambient temperature and atmospheric pressure, where the washing of the leaves was carried out for time periods of 30, 60 and 180 seconds for each leaf sample. Washing was carried out by placing approximately 1 g of each tobacco leaf sample in 25 mL methanol.
(3) Tobacco leaf samples with known low and high BaP content were washed with hexane at ambient temperature and atmospheric pressure, where the washing of the leaves was carried out for time periods of 30, 60, 180 and 300 seconds for each leaf sample, and each wash time was performed for samples in duplicate. Washing was carried out by placing approximately 1 g of each tobacco leaf sample in 25 mL hexane. The exact mass of each leaf sample used is recorded in Table 2 and was used to normalise the absorbance and fluorescence data to 1 g tobacco.

TABLE 2
mass of each leaf sample tested in hexane

| Sample Number | Weight (g) | Shaking time (s) | Hexane volume (mL) |
|---|---|---|---|
| 259-30-A | 0.9994 | 30 | 25 |
| 259-30-B | 1.0486 | | 25 |
| 259-60-A | 1.0307 | 60 | 25 |
| 259-60-B | 0.9761 | | 25 |
| 259-180-A | 1.0563 | 180 | 25 |
| 259-180-B | 1.0571 | | 25 |
| 259-300-A | 1.0944 | 300 | 25 |
| 259-300-B | 1.0061 | | 25 |
| 263-30-A | 1.0656 | 30 | 25 |
| 263-30-B | 1.0236 | | 25 |
| 263-60-A | 1.0053 | 60 | 25 |
| 263-60-B | 1.0474 | | 25 |
| 263-180-A | 1.0011 | 180 | 25 |
| 263-180-B | 1.0162 | | 25 |
| 263-300-A | 1.0115 | 300 | 25 |
| 263-300-B | 1.0250 | | 25 |

### Absorbance Spectrum of BaP

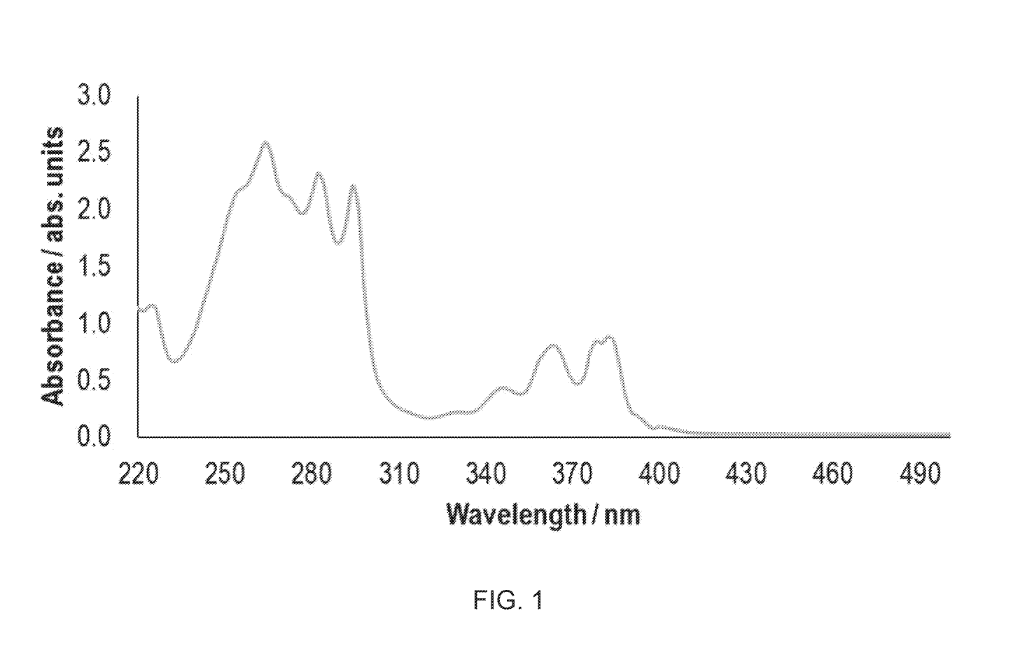

For reference purposes, an absorbance spectrum of 5 ppm BaP was acquired at 2 nm resolution using a Cecil 5000 series dual-beam spectrometer. Methanol was provided as the reference solvent, and solutions were contained in 1 cm path length cuvettes (Starna Scientific Ltd, 23/Q/10, Quartz). The acquired absorbance spectrum of BaP is shown in FIG. 1. The absorbance spectrum of 5 ppm BaP has a maximum at 264 nm, with other maxima at 282 nm, 294 nm, 346 nm, 364 nm and 382 nm. The absorbance spectrum indicates the presence of two electronic bands in BaP (294 nm and 382 nm) with associated vibrational structure.

It was determined from the absorbance spectrum in FIG. 1 that an excitation wavelength of 255 nm or 280 nm would be appropriate for targeting BaP emission.

### Excitation-Emission Matrices of BaP in Methanol and Hexane

Excitation-emission matrices (EEMs) for all samples were acquired using the HORIBA Aqualog® instrument. The HORIBA Aqualog® consists of a UV-enhanced light source, double-grating excitation monochromator and thermoelectrically-cooled CCD detector, which enables simultaneous resolution of full emission spectra at every excitation wavelength. The Aqualog® automatically generates National Institute of Standards and Technology (NIST)-traceable corrected fluorescence spectra. It includes a reference detector, which enables the spectral output of the excitation light to be accounted for by division of the signal EEM by the reference excitation light output. In addition, both the signal and reference detector signals are corrected for the instrument's spectral response using saved spectral correction factors.

For all the EEMs, the Rayleigh lines at λₑₘᵢₛₛᵢₒₙ=λ_{excitation} and 2λₑₘᵢₛₛᵢₒₙ=λ_{excitation} have been removed by setting the data in these regions to zero. In addition, the region of the EEMs where λₑₘᵢₛₛᵢₒₙ<λ_{excitation} has also been set to zero. The Raman line can largely be removed by subtracting the Blank EEM from an EEM of the sample, providing that these EEMs are both acquired at the same instrument settings. All of the EEMs acquired in all of the Examples described herein are shown as Sample EEM−Blank EEM, where Blank EEM is acquired following the excitation and emission of the solvent only.

After subtraction of the Blank EEM and removal of the Rayleigh lines, each EEM was also corrected for any inner filter effects (IFE), which arise when the absorbance of either the excitation or emission light becomes significant. The IFE correction is carried out using the following equation:

IFE=10^{(A}^{ₑₓ}^{-A}^{ₑₘ}^{)/2}

where Aₑₓ is the absorbance at the excitation wavelength and Aₑₘ is the absorbance at the emission wavelength. The absorbance path length is assumed to be 0.5 cm in a 1 cm path length cell. The IFE-corrected fluorescence, F_{corr}, is a product of the measured fluorescence (Fₘₑₐₛ) and the IFE correction (IFE):

F_{corr}=Fₘₑₐₛ×IFE

The IFE correction is greatly simplified with the Aqualog® as the absorbance spectrum of the sample is acquired simultaneously with the EEM.

To enable comparison with literature fluorescence data, a typical method used for standardising fluorescence data is to normalise the fluorescence signal to the fluorescence at an excitation wavelength of 347.5 nm and an emission wavelength of 450 nm from 1 μg/mL (1 mg/L or 1 ppm or 1.28 μM) quinine sulphate dissolved in acid. As used herein, such EEM intensity units are referred to as quinine sulphate units (ppm), QSU (ppm). This enables direct comparison of data collected across different instruments. A certified reference standard of quinine sulphate and acid blank (0.105M perchloric acid) was purchased from Starna Scientific Ltd. (RM-QS00, set serial number 19855, cell serial number 43084, blank serial number 43052, certificate number 97291, certificate date Aug. 9, 2014), with both solutions provided in 1 cm path length fluorometer cells composed of Spectrosil Quartz.

With the exception of the EEM of 296 nM BaP dissolved in methanol (see below), all the EEMs acquired in each of the Examples described herein were normalised by dividing the EEM by the fluorescence intensity which would be recorded from 1 ng/mL (1 ppb) quinine sulphate at an excitation wavelength of 347.5 nm and an emission wavelength of 450 nm. A minimum of five repeats of the 1 ppm quinine sulphate emission spectrum at an excitation wavelength of 347.5 nm were acquired on each day of EEM acquisition, and the EEMs acquired on that day were divided by the average emission at 450 nm divided by 1000 to convert from ppm to ppb quinine sulphate. The resultant EEM intensity units are referred to as quinine sulphate units, QSU.

Excitation wavelength increments of 1 nm in the range 200-500 nm were used to collect emission spectra in the range 250-800 nm with 1.12 nm increments. For direct leaf analysis, an integration time of 0.05 s was used, while for the remainder of the EEMs an integration time of 0.3 s was used. From this data, a Python script was written to apply the quinine sulphate correction factor and plot the excitation-emission matrices in the range 240-480 nm excitation and 250-550 nm.

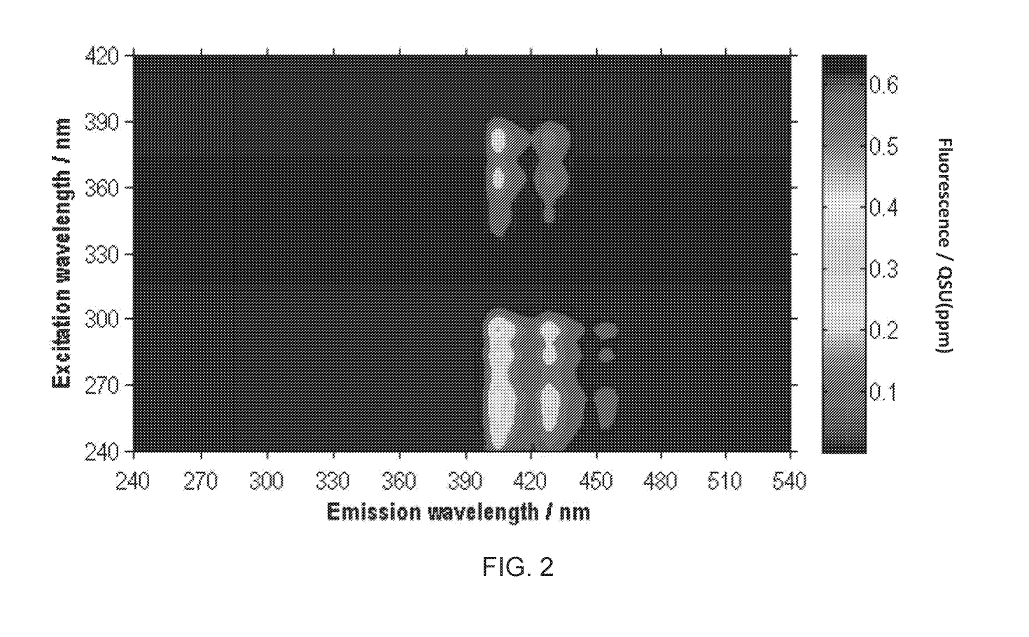

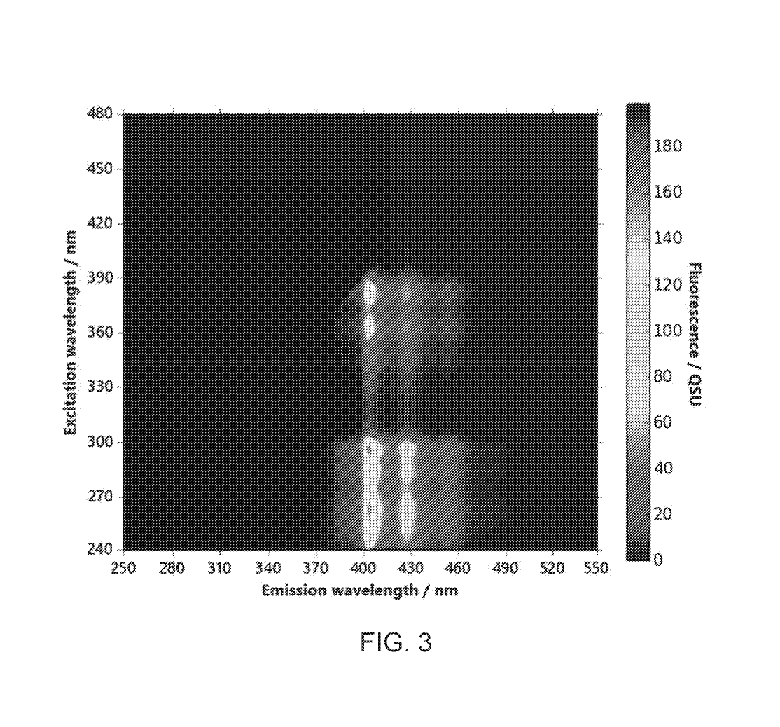

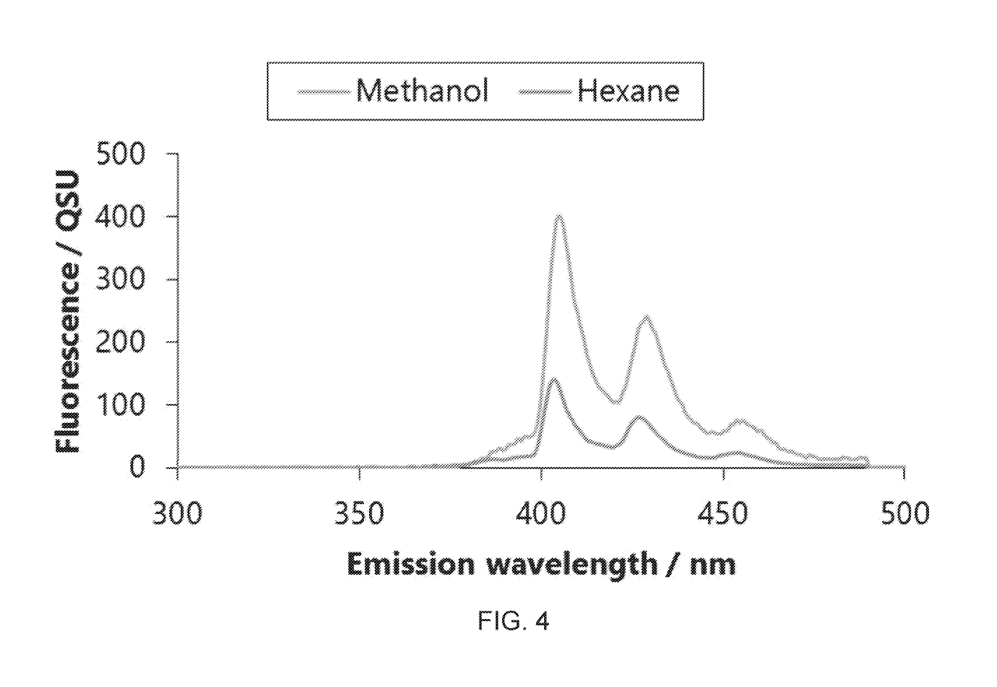

The EEMs of 396 nM BaP dissolved in methanol and hexane are shown in FIGS. 2 and 3, respectively. In each of the EEMs, the fluorescence intensity is normalised to 100 nM BaP, and are shown in units of QSU. As indicated above, the fluorescence intensity in the EEM of 396 nM BaP dissolved in methanol is shown as normalised to the fluorescence at an excitation wavelength of 347.5 nm and an emission wavelength of 450 nm from 1 μg/mL (1 mg/L or 1 ppm or 1.28 μM) quinine sulphate dissolved in acid, but without converting the units from ppm to ppb quinine sulphate. In other words, the fluorescence intensity in the EEM of FIG. 2 is shown in units of QSU (ppm). A comparison of the fluorescence emission spectra in methanol and hexane extracted from the EEMs at a fixed excitation wavelength of 255 nm is shown in FIG. 4, with the fluorescence intensity for both spectra in methanol and hexane shown in units of QSU to enable a direct comparison. It can be seen from the results in FIG. 4 that the fluorescence in methanol is approximately 2.9 times stronger than that in hexane. The peak positions are shifted to shorter wavelengths by less than 2.5 nm in hexane compared to methanol. It is clear from FIG. 4 that there are two peak emission wavelengths in the fluorescence spectrum for BaP in methanol: ˜405 nm and ˜429 nm. These peaks are shifted to slightly shorter wavelengths for BaP in hexane.

### (1) Direct Leaf Analysis (Comparative Analysis)

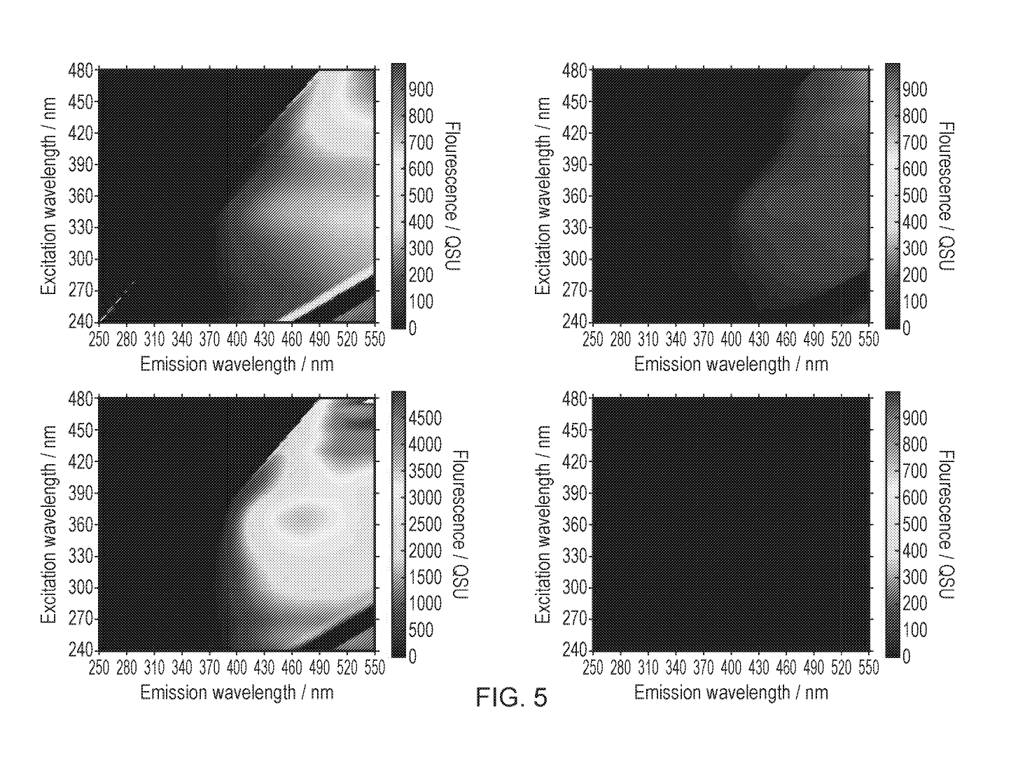

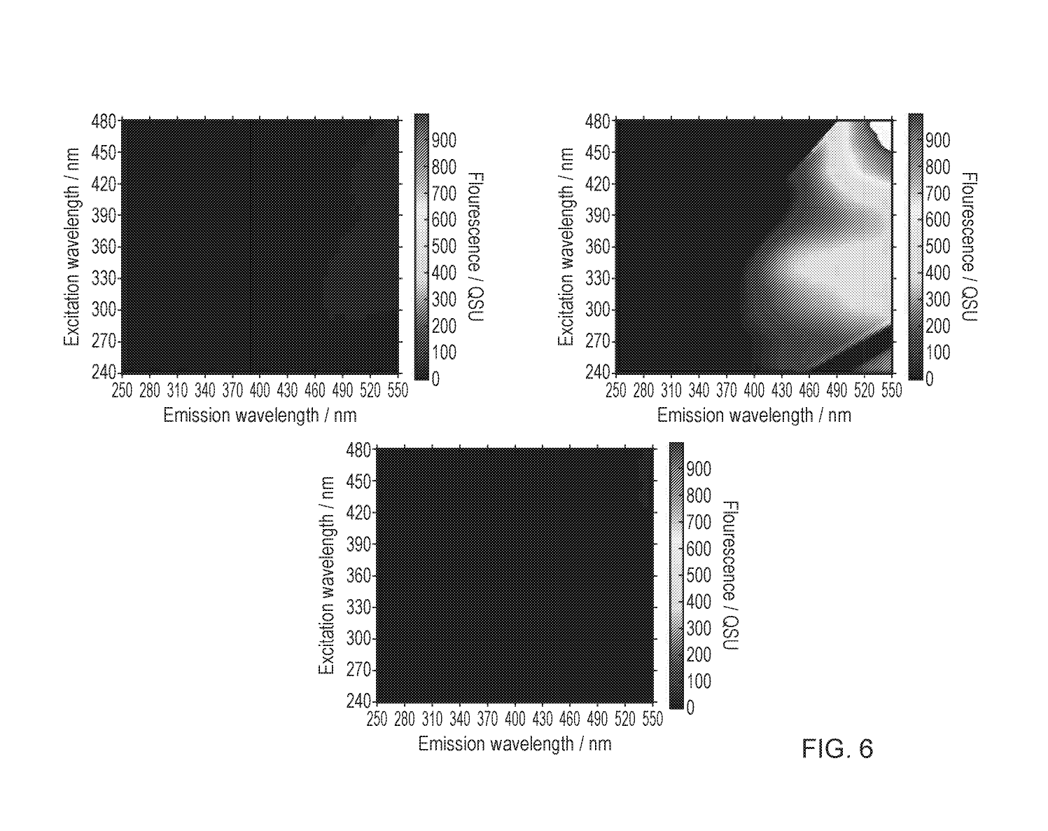

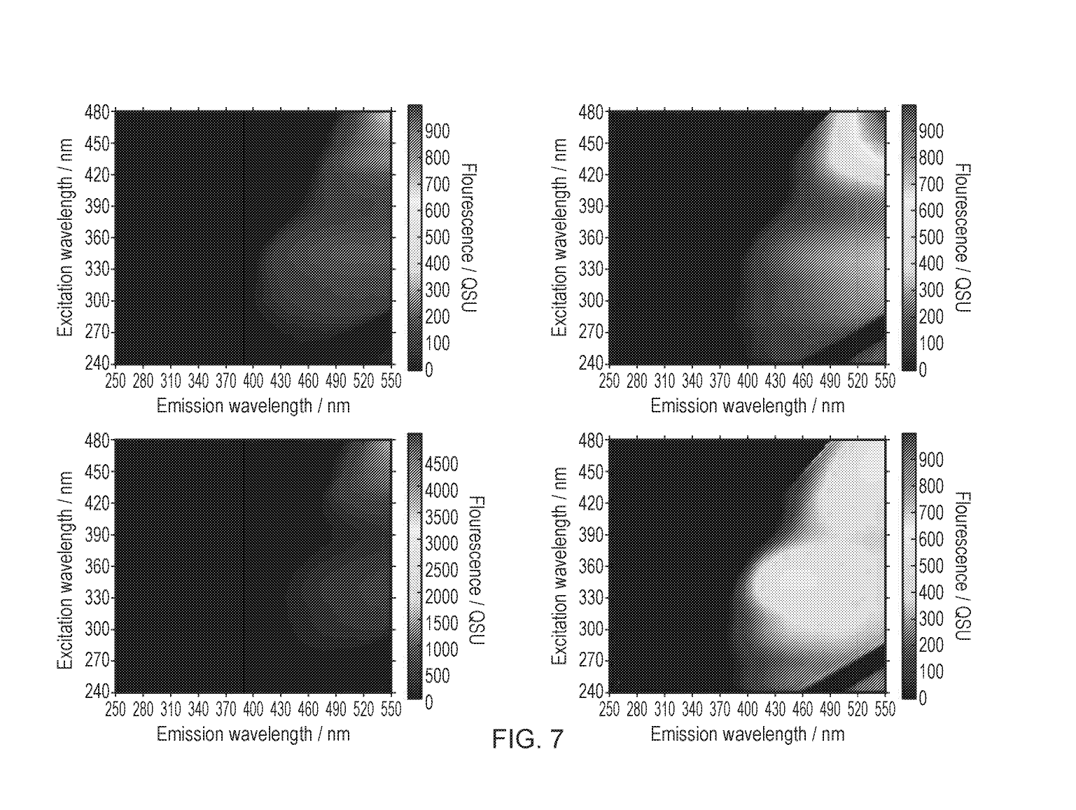

EEMs were acquired by direct analysis of the low, medium and high BaP content at different areas of the surface of the tobacco leaves for each sample. The results for direct leaf analysis for low (sample 259), medium (sample 261) and high (sample 263) BaP content leaves are shown in FIGS. 5, 6 and 7, respectively. Four repeats of different leaf areas are shown in FIGS. 5 and 7 for the low (sample 259) and high (sample 263) BaP content leaves. Three repeats of different leaf areas are shown in FIG. 6 for medium (sample 261) BaP content leaves. It is clear from each of these Figures that measuring the excitation-emission spectra of the leaves directly (i.e. without first washing them in solvent) is not a suitable method for assessing BaP content. No distinctive BaP fluorescence is recorded in any of the direct EEMs, and there is a large and varying fluorescence background observed both for leaf samples having the same nominal BaP content, and for leaf samples having different BaP contents. This indicates that the leaf is contributing significantly to the fluorescence recorded in the direct EEM approach, and that direct leaf analysis is therefore not suitable for assessing BaP contamination levels in the tobacco leaf samples.

### (2) Tobacco Leaf Samples Washed in Methanol (Comparative Analysis)

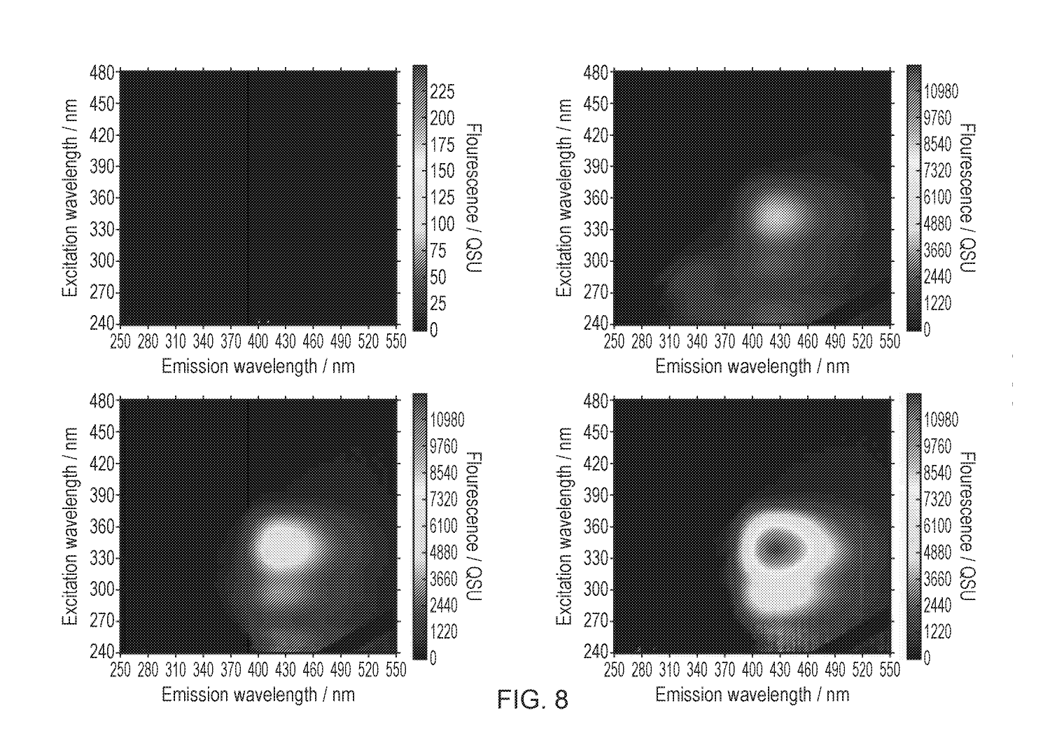

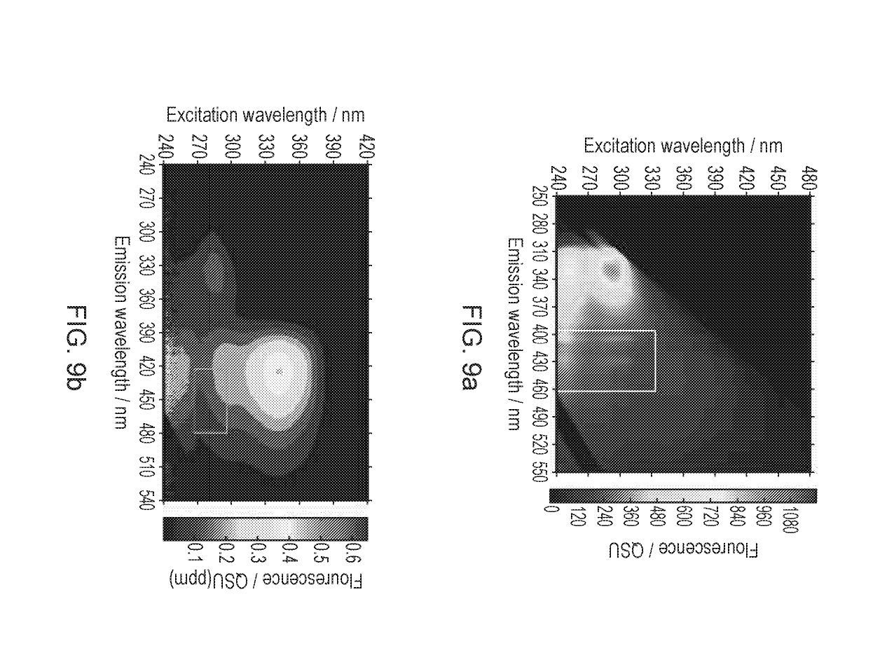

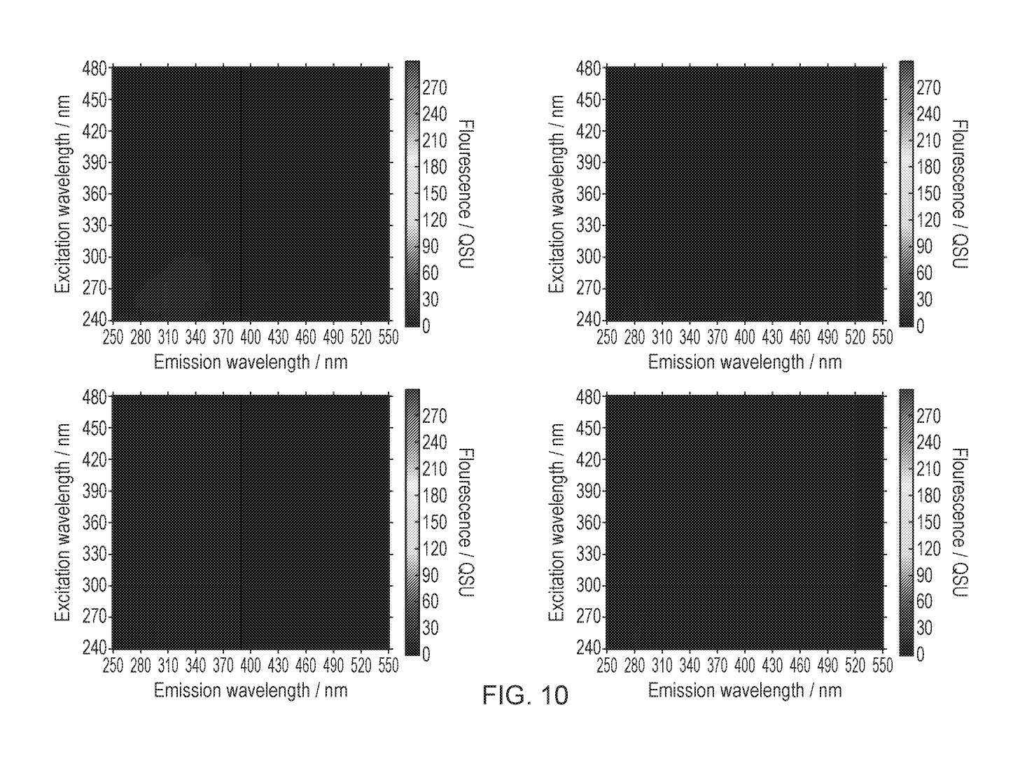

EEMs were acquired for tobacco leaf samples washed in methanol at wash times of 30 s, 60 s and 180 s. The EEMs for blank (i.e. methanol only) (top left), 30 s (top right), 60 s (bottom left) and 180 s (bottom right) methanol leaf wash samples for high BaP content leaves are shown in FIG. 8.

No distinctive BaP fluorescence at the expected emission wavelengths can be seen in FIG. 8 suggesting that there is a relatively large and varying fluorescence background due to the extraction of the tobacco leaf pigments by the methanol solvent.

It is evident from FIG. 8 that, even with reducing the leaf wash times to between 30 and 180 seconds, no distinctive BaP fluorescence is observed. Since it is not practical to reduce the leaf wash times to less than 30 seconds, these results indicate that methanol, when used as the sole solvent, is not a suitable solvent for washing off the surface BaP contamination without also extracting the pigments in the leaf.

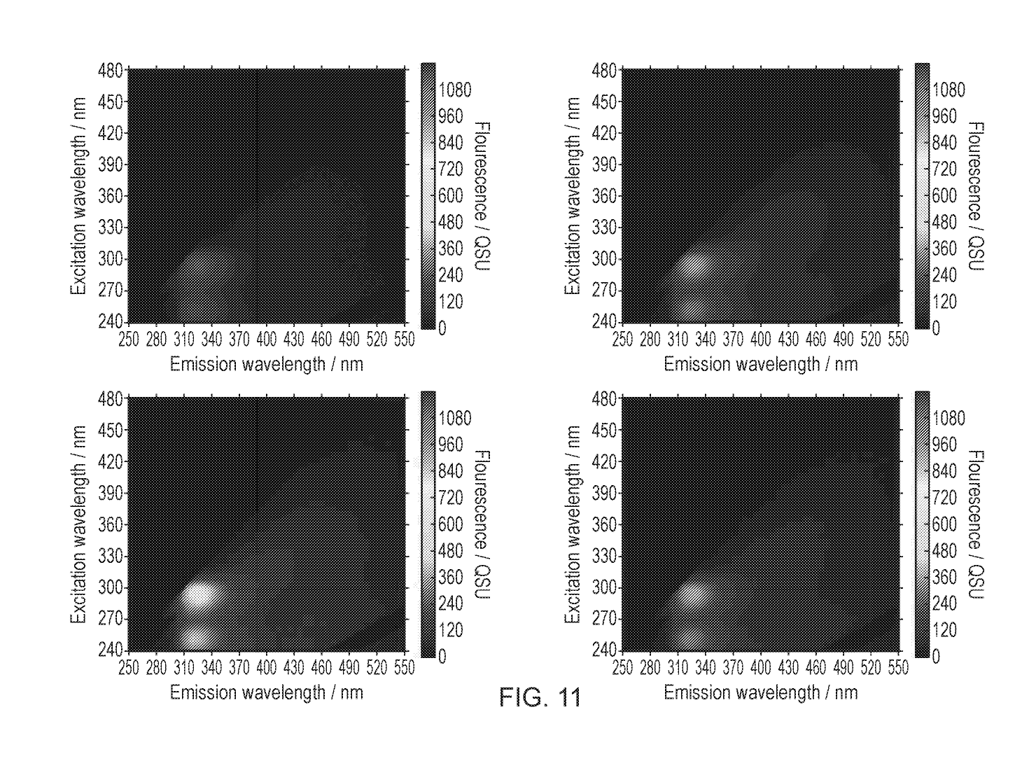

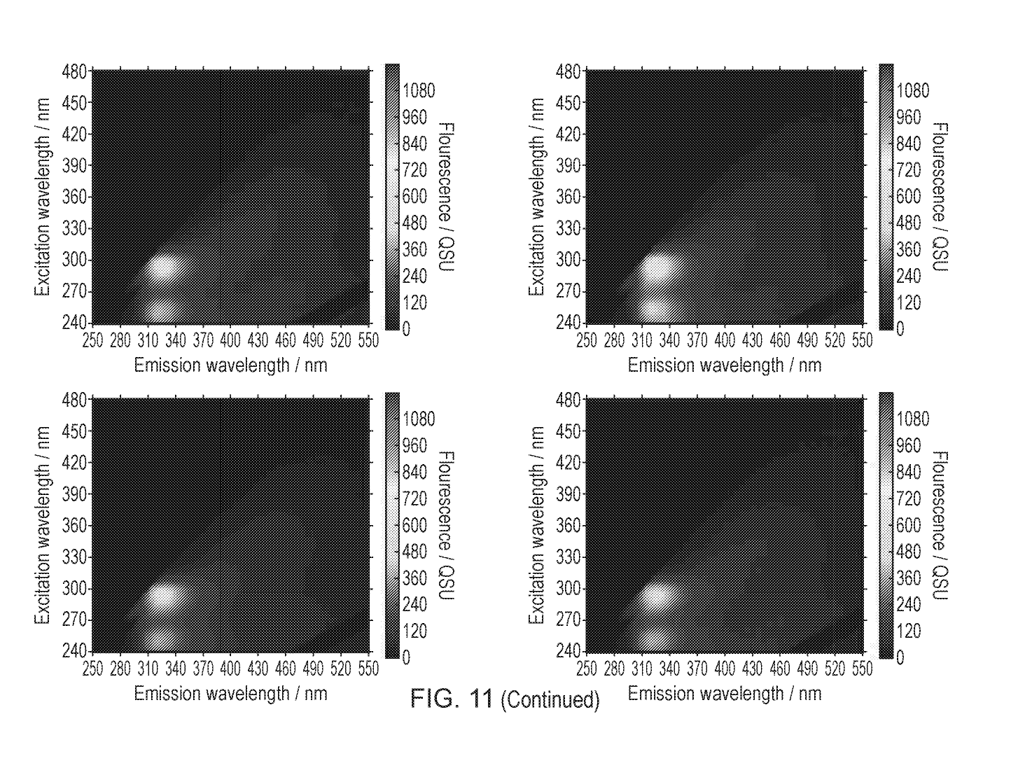

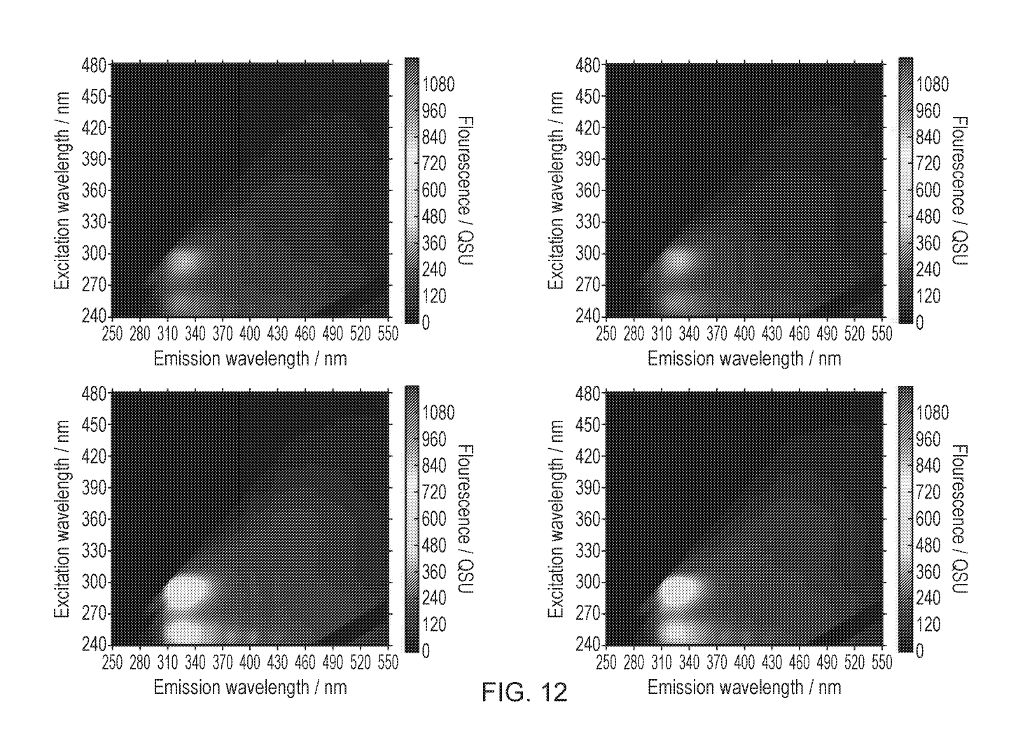

This can be further seen by a comparison of the EEMs obtained by washing sample 263 (high BaP content) in either methanol for 60 seconds or hexane for a period of 300 seconds. The EEM acquired by washing this sample 263 in hexane for 300 seconds is shown in FIG. 9a, and the EEM acquired by washing this sample 263 in methanol for 60 seconds is shown in FIG. 9b. It can be seen that BaP signals are clearly visible within the white frame shown in the EEM of the sample shown in FIG. 9a, whilst no BaP signals can be seen in FIG. 9b. The orange rectangle in FIG. 9b denotes the region in which BaP signals should be observed, but they are obscured by fluorescence from co-extracted substances. These results further indicate that methanol, when used as the only solvent, is not a suitable solvent for washing off the surface BaP contamination without also extracting the pigments in the leaf.

### (3) Tobacco Leaf Samples Washed in Hexane

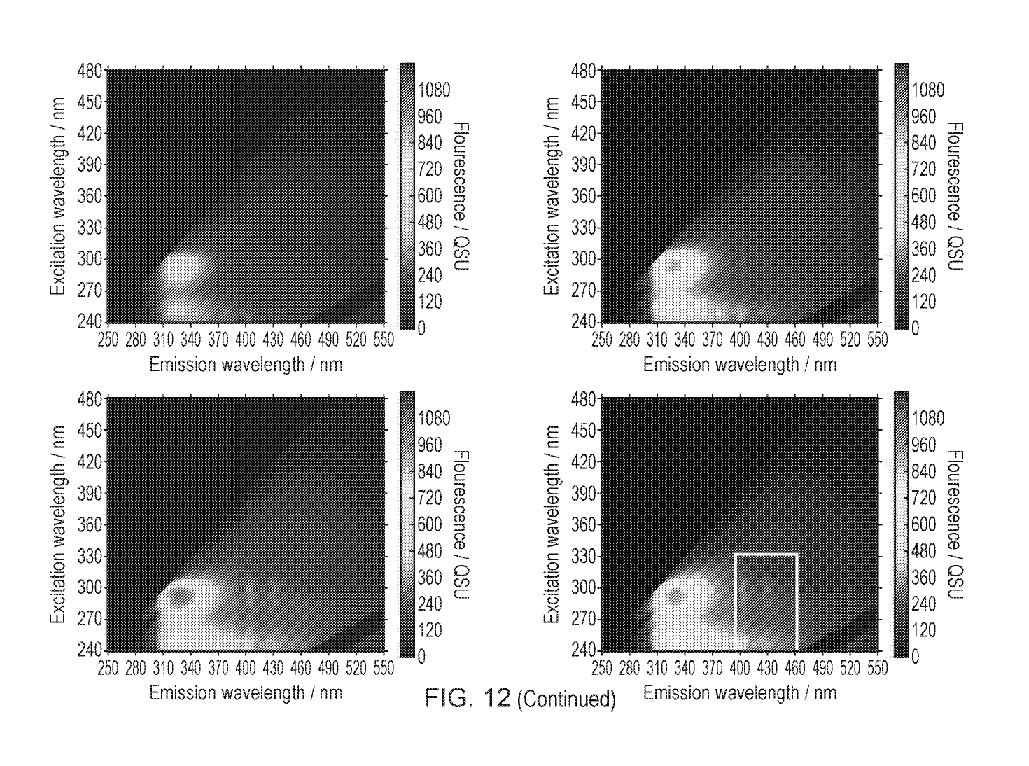

The blank EEMs (i.e. hexane only) for the four wash times assessed for tobacco leaf samples washed in hexane (30 s, 60 s, 180 s, 300 s) were acquired, and the results shown in FIG. 10. The EEM for 30 s hexane wash is shown top left, the EEM for 60 s hexane wash is shown top right, the EEM for 180 s hexane wash is shown bottom left and the EEM for 300 s hexane wash is shown bottom right in FIG. 10. No discernible fluorescence is detected.

The EEMs for the low BaP content leaf samples (sample 259) were acquired in duplicate for the four leaf wash times assessed, and are presented in FIG. 11. The EEMs of the samples in group A (see identification in Table 2) are shown on the left and the EEMs of the samples in group B are shown on the right of FIG. 11. The EEMs in the top row are for 30 s leaf wash, the EEMs in the second row are for 60 s leaf wash, the EEMs in the third row are for 180 s leaf wash, and the EEMs in the bottom row are for 300 s leaf wash. Increasing levels of fluorescence between 310-340 nm emission wavelengths were observed with increasing leaf wash times. There was no discernible evidence of any BaP fluorescence, which would be present at emission wavelengths above 400 nm.

The EEMs for the high BaP content leaf samples (sample 263) were acquired in duplicate for the four leaf wash times assessed, and are presented in FIG. 12. The EEMs of the samples in group A are shown on the left and the EEMs of the samples in group B are shown on the right of FIG. 12. The EEMs in the top row are for 30 s leaf wash, the EEMs in the second row are for 60 s leaf wash, the EEMs in the third row are for 180 s leaf wash, and the EEMs in the bottom row are for 300 s leaf wash. The BaP region is marked as a white rectangle in FIG. 12 for reference. As for the low BaP content samples, increasing levels of fluorescence between 310-340 nm were observed with increasing leaf wash times. However, in FIG. 12, it can be seen that there is also structured fluorescence observed above 400 nm that appears characteristic of BaP, as is evident from a comparison of FIG. 3 with FIG. 12. The data shown in FIGS. 11 and 12 indicate that it is possible to visually identify from the EEM which of the tobacco leaf samples washed in hexane has low or high BaP content.

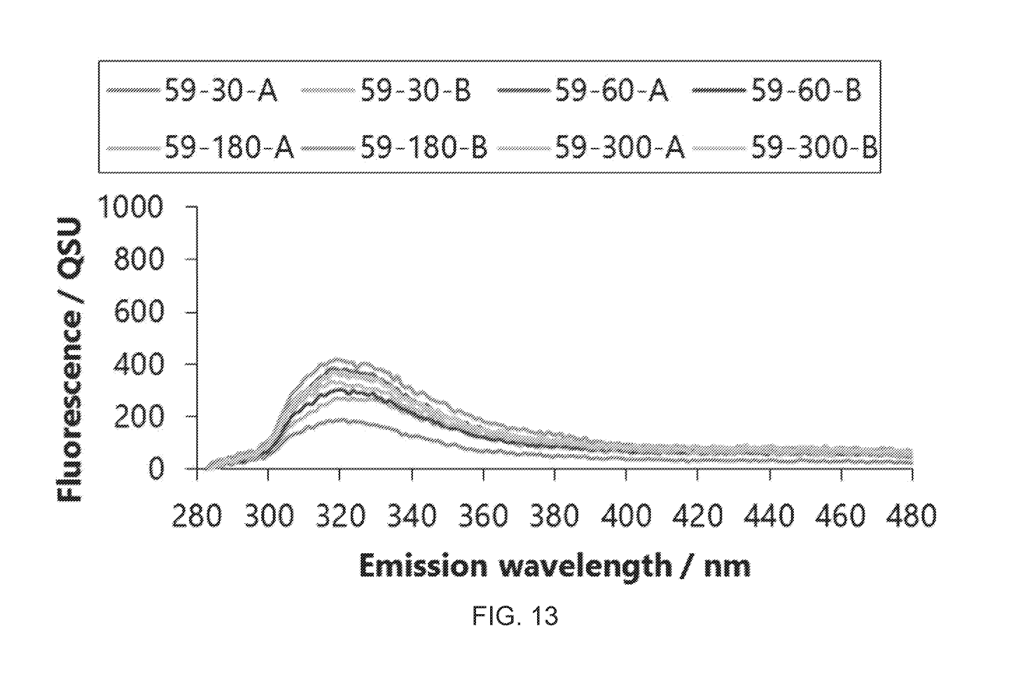

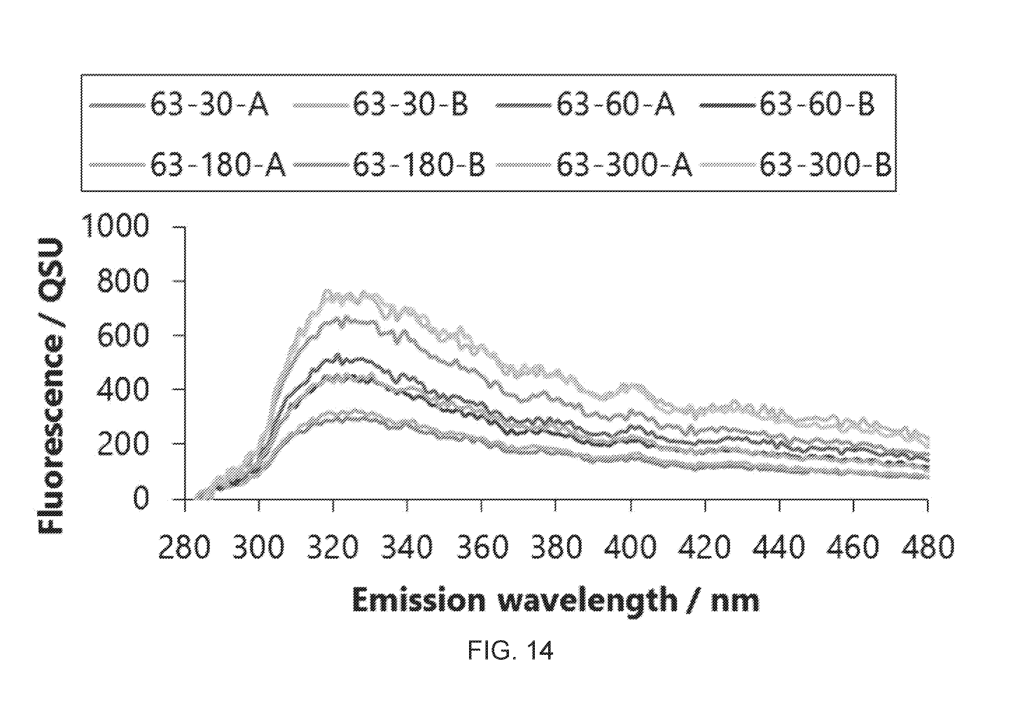

To estimate the detection limit, the emission spectra at 255 nm excitation was extracted from each EEM and are shown in FIGS. 13 and 14 for the low and high BaP content leaf wash samples, respectively.

From the emission spectra at 255 nm excitation, a potential sensor view was established by calculating the average fluorescence at one or two emission wavelengths for the different leaf wash times. This analysis was completed for 330 nm and 400 nm emission wavelengths, with full-width half-maximum (FWHM) bandpass regions of 1, 10, 20, 30, and 50 nm. These wavelengths were chosen since 330 nm is close to the emission maximum of the short wavelength fluorescence observed in both low and high BaP content leaf wash samples, and 400 nm is close to the emission maximum of BaP and is present at elevated levels only in the high BaP content leaf wash samples. The bandpass is a compromise between detecting too low a level of fluorescence and/or being sensitive to single nanometer shifts in fluorescence, and detecting too wide a region of fluorescence so that the background fluorescence leaks into the BaP fluorescence detection region.

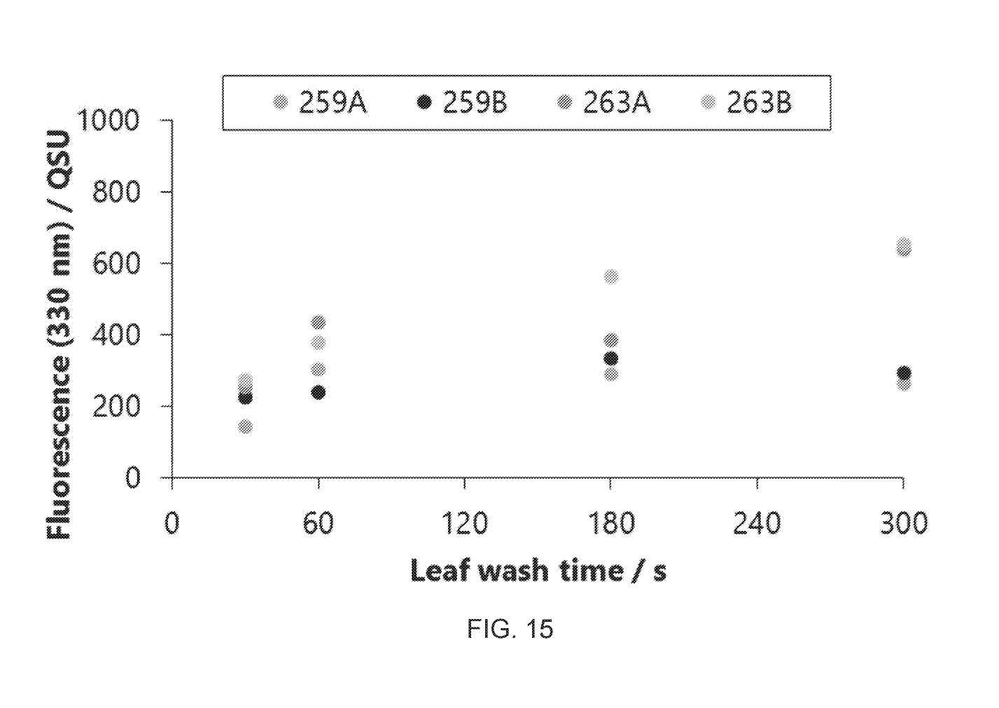

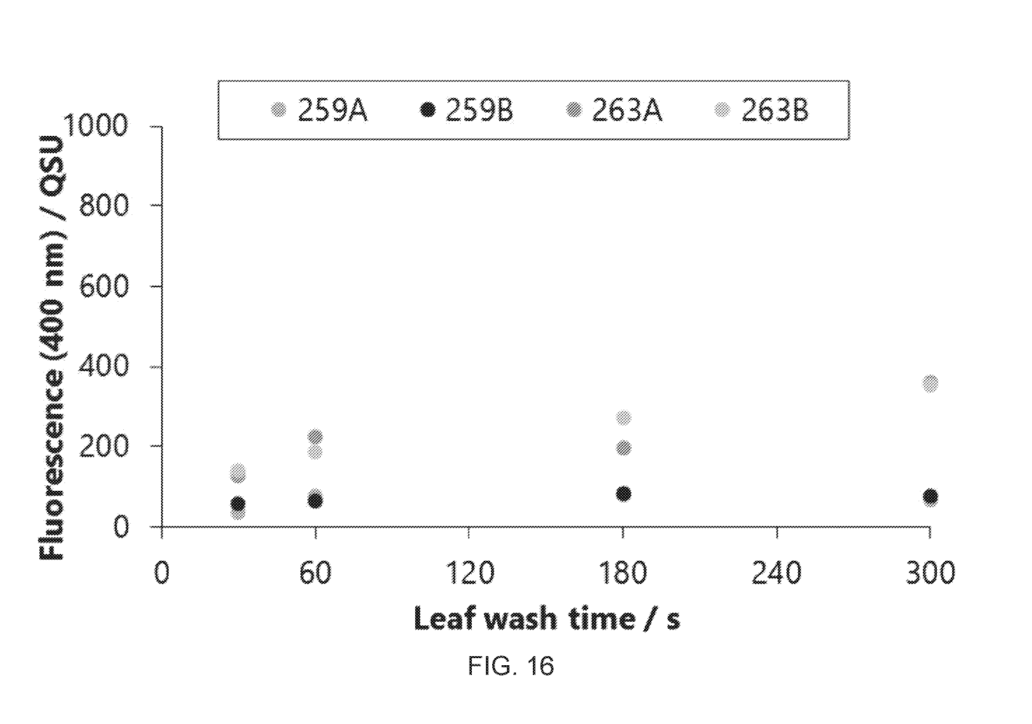

An emission bandpass of ±5 nm was found to be optimum and the average fluorescence at an emission peak of 330 nm with a 10 nm (±5 nm) bandpass at varying leaf wash times is shown in FIG. 15, and the average fluorescence at an emission peak of 400 nm with a 10 nm (±5 nm) bandpass at varying leaf wash times is shown in FIG. 16.

The fluorescence at both 330 nm and 400 nm increases with leaf wash time, and is consistently higher for the high than for the low BaP content leaf wash sample.

However, below 300 seconds in particular, there is no significant difference between the fluorescence intensity of the two BaP content samples (see FIGS. 15 and 16). In other words, there is not a significant difference in fluorescence intensity to accurately distinguish between a high level of BaP and a low level of BaP when using a single fluorescence peak at below 300 seconds.

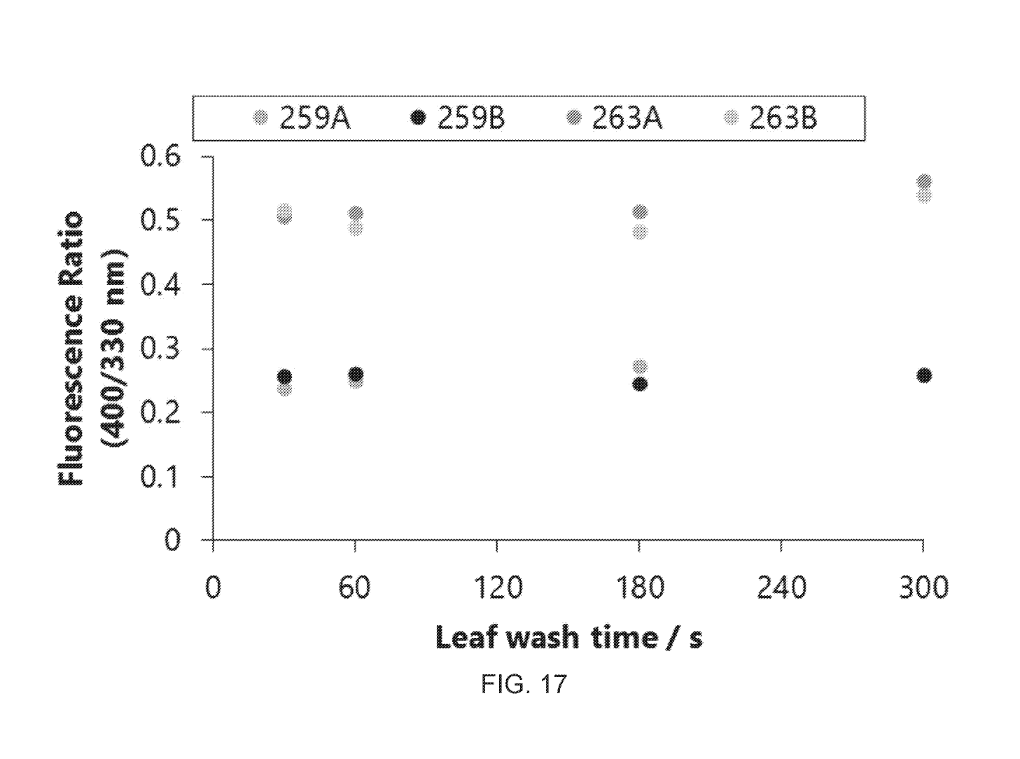

The 400/330 nm fluorescence ratio (i.e. the ratio of the fluorescence intensity of the peak at 400 nm to the fluorescence intensity of the peak at 330 nm in the emission spectrum) was calculated for the different leaf wash times, and is plotted in FIG. 17. The fluorescence ratio at 400 nm to that at 330 nm presented in FIG. 17 provided significantly more stable results compared with the average fluorescence at a single fluorescence peak both across the four different leaf wash times and between the two duplicate samples at each leaf wash time. The fluorescence ratio (400/330 nm), therefore, provides an extraction time-independent method (up to tested 5 minutes) of assessing BaP leaf content.

Determining the average fluorescence ratio for the low BaP content leaf samples, and determining the limit of detection as three times the standard deviation above the blank, provides an estimated limit of detection of 10.0 nM/L (2.52 μg BaP/ml) BaP via dual-wavelength fluorescence at 255 nm excitation. This was deemed sufficient to determine whether a leaf sample with unknown BaP content has a low, medium, or high level of BaP.

The results at 255 nm excitation for the range of bandpass regions analysed are presented in Table 3, showing that 10 nm bandpass provides the lowest detection limit for the current data set.

TABLE 3
estimated detection limits for different bandpass values

| | | | Estimated |
|---|---|---|---|
| Emission | Emission | | limit of |
| wavelength 1 | wavelength 2 | Bandpass | detection |
| (nm) | (nm) | (nm) | (nM/L BaP) |
| 330 | 400 | 1 | 13.5 |
| | | 10 | 10.0 |
| | | 20 | 12.2 |
| | | 30 | 12.5 |
| | | 40 | 14.3 |

The effect of moving to longer wavelength fluorescence was also assessed, for example by moving the second emission wavelength from 400 to 430 nm. However, this caused the detection limit to worsen from an estimated 10.0 nM to 21.3 nM.

While the bench-top EEMs are automatically corrected for absorbance, the additional benefit of the ratio at a single excitation wavelength is that the fluorescence ratio becomes, to a certain extent, absorbance-independent, since the majority of the absorbance occurs at the excitation wavelength and the two fluorescence emission regions are excited at the same excitation wavelength.

The present inventors have thus found that the EEMs of the tobacco leaf wash samples are correlated to BaP content, particularly for those samples washed in hexane. Furthermore, it has been shown that the fluorescence ratio at 400/330 nm emission at 255 nm excitation is independent of extraction time up to a wash time of 5 minutes.

### Example 2

Six tobacco leaf samples were selected having known contents of BaP, which are defined as either low, medium or high levels of BaP. Approximately 1 g of each tobacco leaf sample was placed in 25 mL hexane, and washed for a period of time of 180 s. Six tobacco samples were selected for analysis, as shown in Table 4. These samples were prepared in order to be used as calibration samples. It is noted that low BaP content sample 259 and high BaP content sample 263 were investigated when washed in hexane in Example 1.

TABLE 4
cured tobacco leaf samples having known BaP content

| Sample | | BaP content | BaP content | BaP content |
|---|---|---|---|---|
| Number | Weight (g) | (ng g⁻¹) | (ppb g⁻¹) | (nM g⁻¹) |
| 259 | 100 | 3.85 | 0.154 | 0.610 |
| 258 | 100 | 50.38 | 2.015 | 7.987 |
| 262 | 100 | 153.11 | 6.124 | 24.273 |
| 261 | 100 | 256.91 | 10.276 | 40.729 |
| 260 | 100 | 451.67 | 18.067 | 71.606 |
| 263 | 50 | 509.64 | 20.386 | 80.796 |

Samples 259 and 258 are considered to have a low level of BaP, samples 262 and 261 are considered to have a medium level of BaP, and samples 260 and 263 are considered to have a high level of BaP.

The washing of the samples in hexane for 180 s in each case caused the removal of less than 5 wt. % BaP from the surface of the tobacco leaf, as is shown in Table 5 below. Table 5 shows a comparison of BaP concentration levels generated by using exhaustive extraction followed by Gas Chromatography Mass Spectrometry (GC/MS) analysis (see column 3 entitled “GC/MS Total BaP”) and the concentration levels measured in leaf wash extracts by Gas Chromatography High Resolution Mass Spectrometry (GC/HR-MS) analysis (see column 2 entitled “PCA GC/HR-MS Leaf Wash Samples”). The percentage ratio of BaP content from leaf wash samples compared to the total BaP in the tobacco leaves is shown in column 4 of Table 5:

TABLE 5
percentage weight of BaP removed from tobacco leaves

| | GC/HR-MS | PCA GC/MS | Ratio Leaf |
|---|---|---|---|
| Tobacco | Leaf wash samples | Total B[a]P | wash/total |
| sample | B[a]P (ng/g leaf) | B[a]P (ng/g leaf) | R[a]P (%) |
| LF 14/00259 | 0.16 | 3.85 | 4.2 |
| | 0.14 | 3.85 | 3.6 |
| | 0.10 | 3.85 | 2.6 |
| LF 14/00258 | 0.16 | 50.38 | 0.3 |
| | 0.13 | 50.38 | 0.2 |
| | 0.10 | 50.38 | 0.2 |
| LF 14/00262 | 0.60 | 153.11 | 0.4 |
| | 0.44 | 153.11 | 0.3 |
| | 0.06 | 153.11 | 0.04 |
| LF 14/00261 | 1.45 | 256.91 | 0.6 |
| | 1.70 | 256.91 | 0.7 |
| | 1.11 | 256.91 | 0.4 |
| LF 14/00260 | 2.71 | 451.67 | 0.6 |
| | 2.85 | 451.67 | 0.6 |
| | 6.24 | 451.67 | 1.4 |
| LF 14/00263 | 6.72 | 509.64 | 1.3 |
| | 5.85 | 509.64 | 1.1 |
| | 4.00 | 509.64 | 0.8 |

As shown in Table 5, the total amount of BaP washed from the surface of the tobacco leaves using hexane and a wash time of 180 s was in the range of from 0.04 to 4.2 wt. %.

The exact mass of each leaf sample used is shown in Table 6. It is worth noting that this mass data was not used as part of the data processing, since the fluorescence ratios being examined are independent of the precise mass used. Three replicates of each leaf sample were generated and analysed.

TABLE 6
mass of each leaf sample tested in hexane

| Sample | Mass replicate 1 (g) | Mass replicate 2 (g) | Mass replicate 3 (g) |
|---|---|---|---|
| 258 | 0.8988 | 0.8792 | 0.9932 |
| 259 | 0.8915 | 0.9024 | 0.8401 |
| 260 | 0.9054 | 0.8508 | 0.8373 |
| 261 | 0.8860 | 0.8896 | 0.9464 |
| 262 | 0.9533 | 0.8453 | 0.9162 |
| 263 | 1.1768 | 0.9813 | 1.2855 |

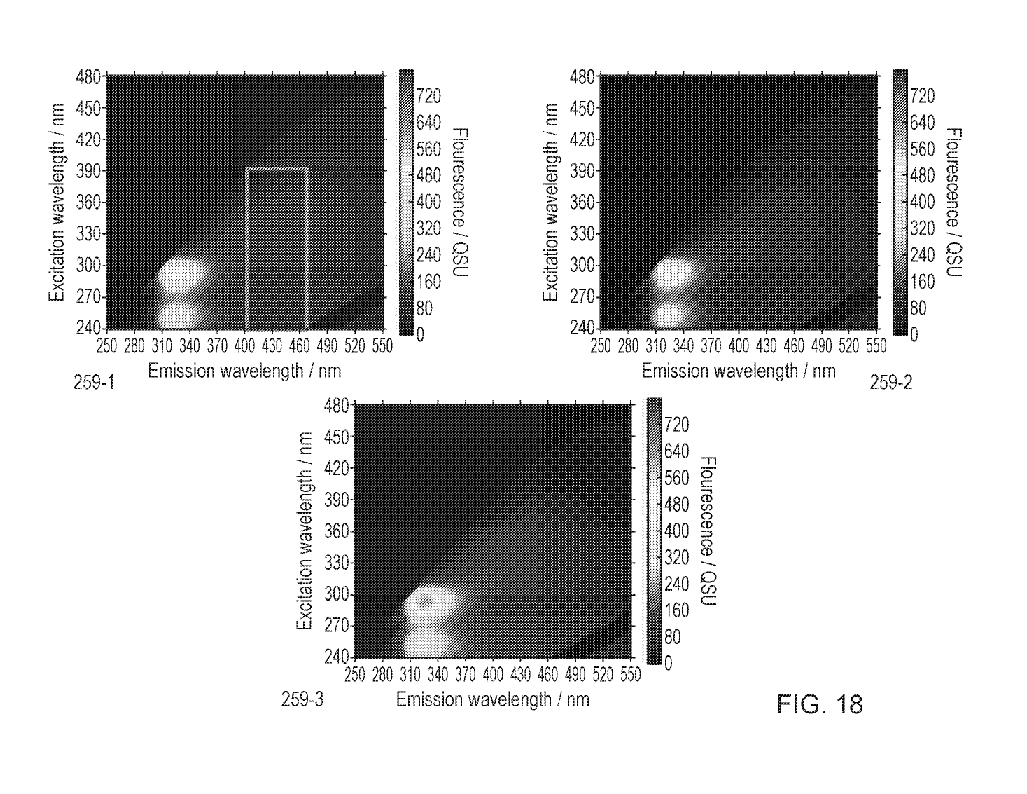

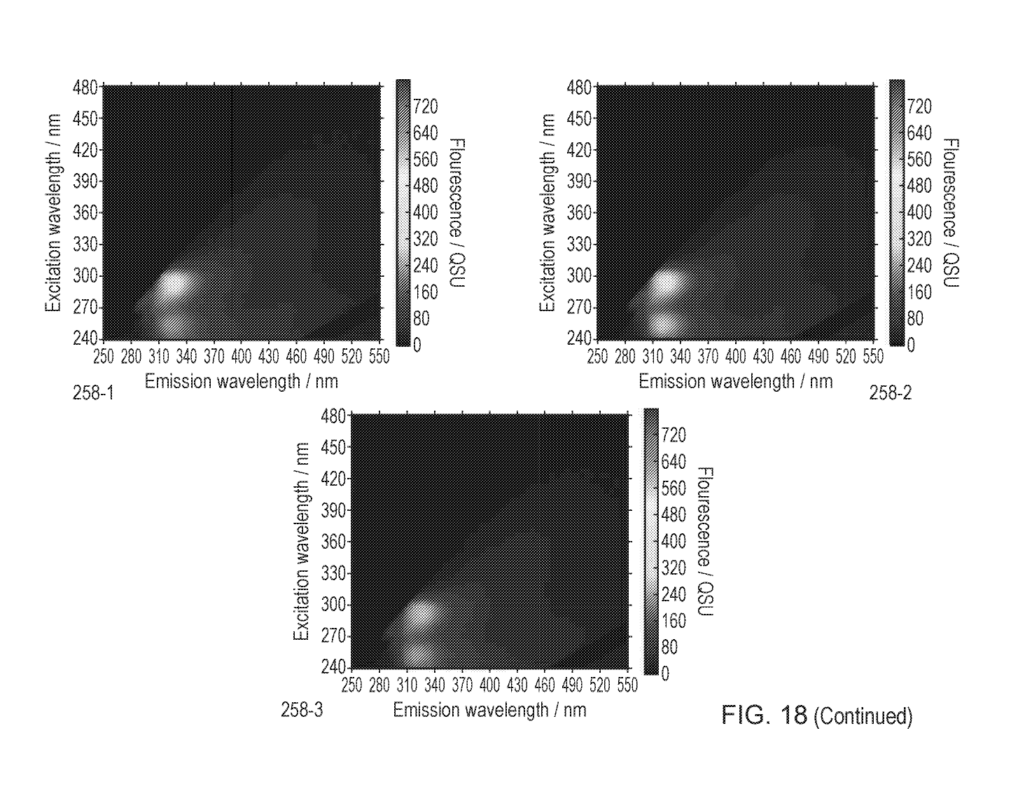

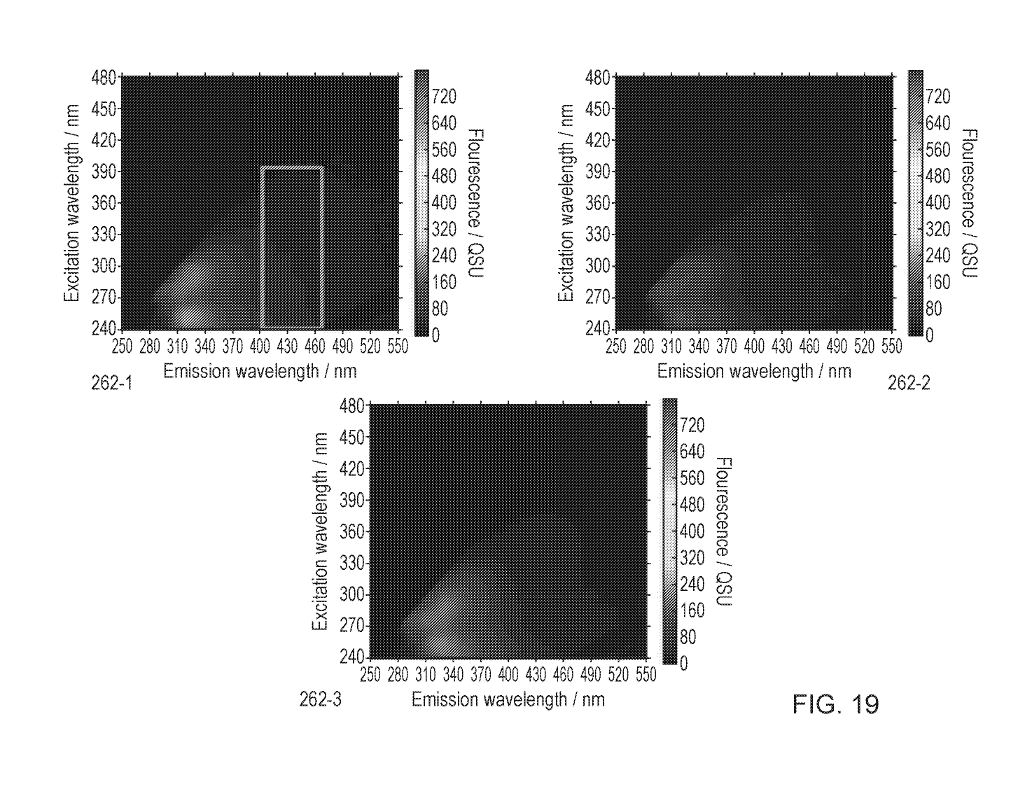

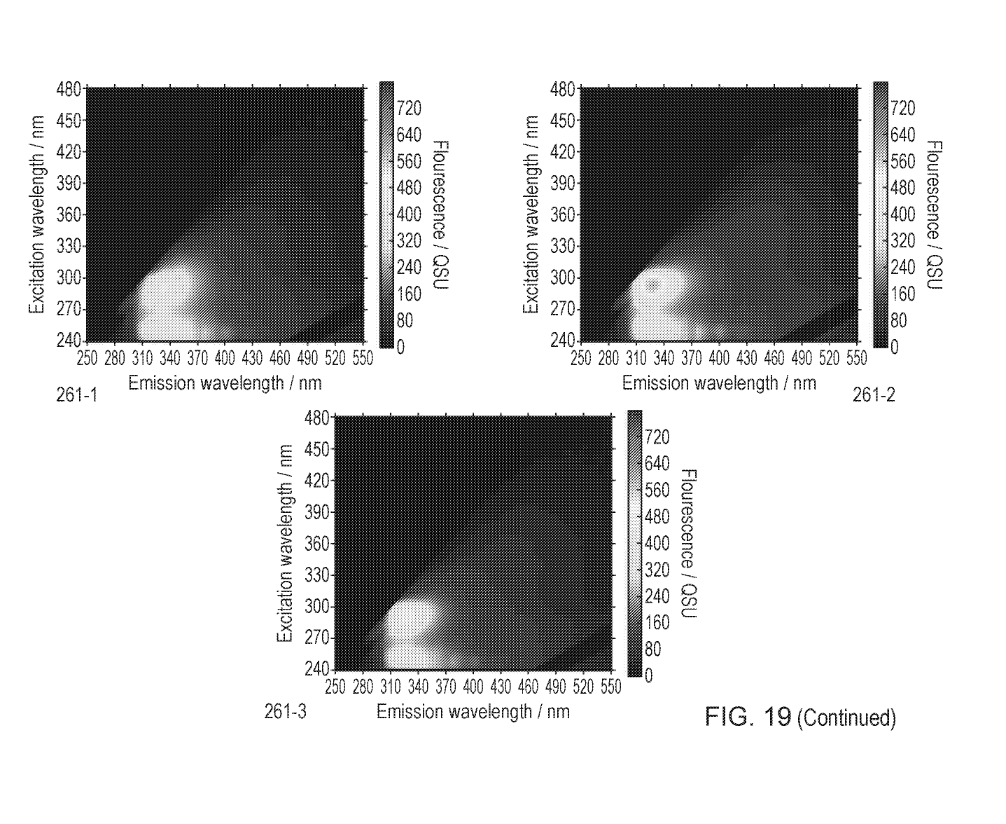

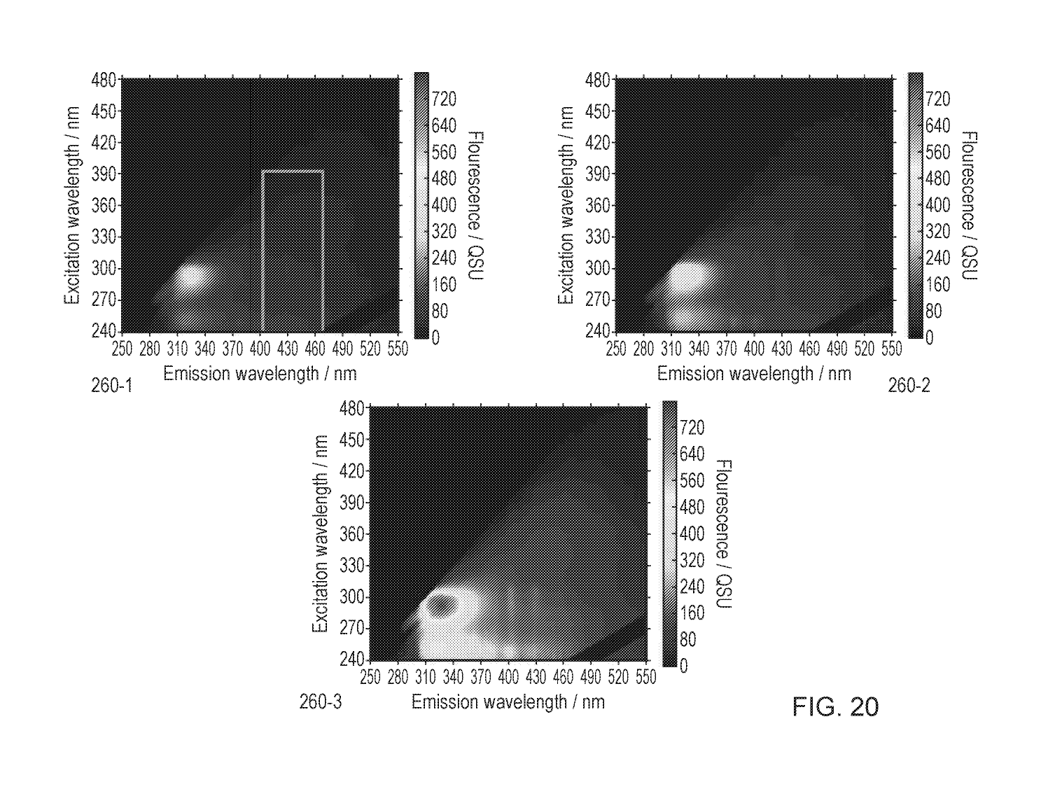

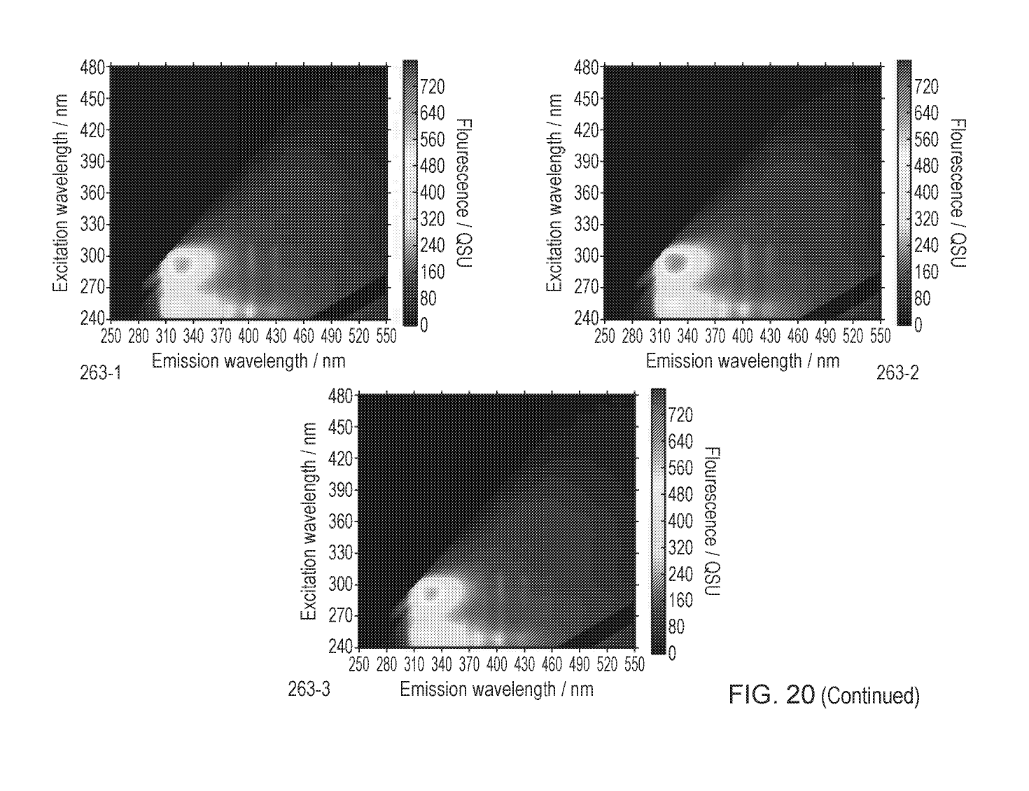

EEMs were acquired using the HORIBA Aqualog® following the procedure described in Example 1. The EEMs for each of the low BaP content samples are shown in FIG. 18, the EEMs for each of the medium BaP content samples are shown in FIG. 19, and the EEMs for each of the high BaP content samples are shown in FIG. 20. The approximate region of BaP fluorescence has been marked as a rectangle on the first EEM in each Figure for reference. It can be seen from FIGS. 18 to 20 that BaP fluorescence is visible in the high and medium BaP content samples, and is absent from the low BaP content samples. It can also be seen that the level of background UV fluorescence, observed at emission wavelengths of approximately 300-370 nm, is highly variable between the different leaf samples.

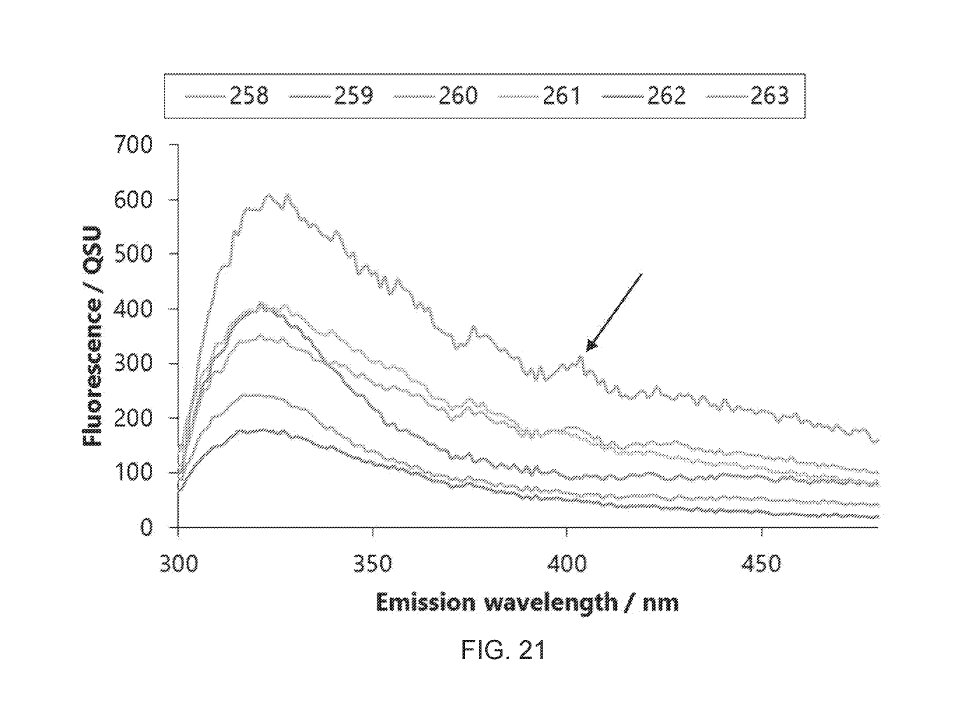

The average emission spectrum for each leaf wash sample at 255 nm excitation was extracted. The resulting emission spectra for each leaf wash sample are shown in FIG. 21, demonstrating the variability in leaf background fluorescence across the different leaf wash samples. The peak at 405 nm (which is indicative of the presence of BaP) is marked with an arrow in FIG. 21. The presence of BaP can be seen at around 400 nm in samples 263 and 260 (high BaP content).

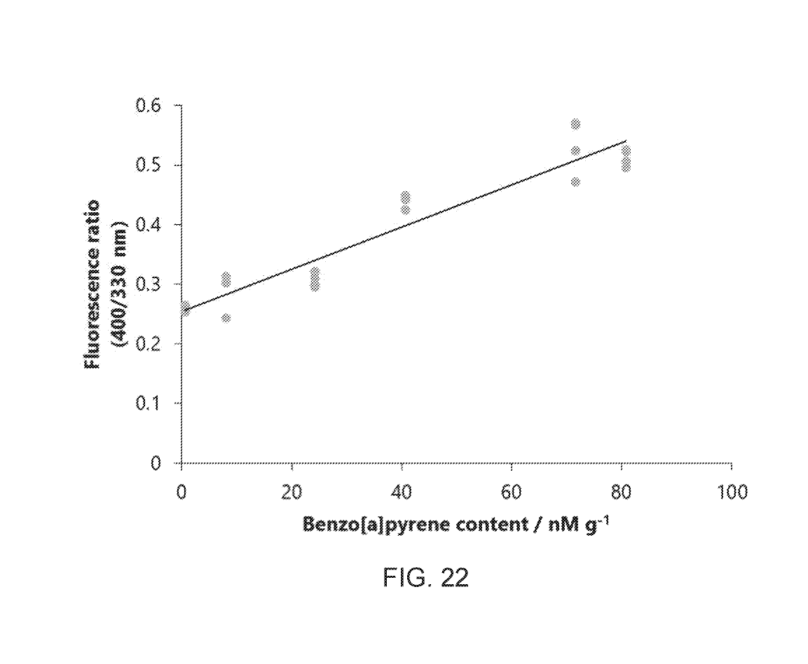

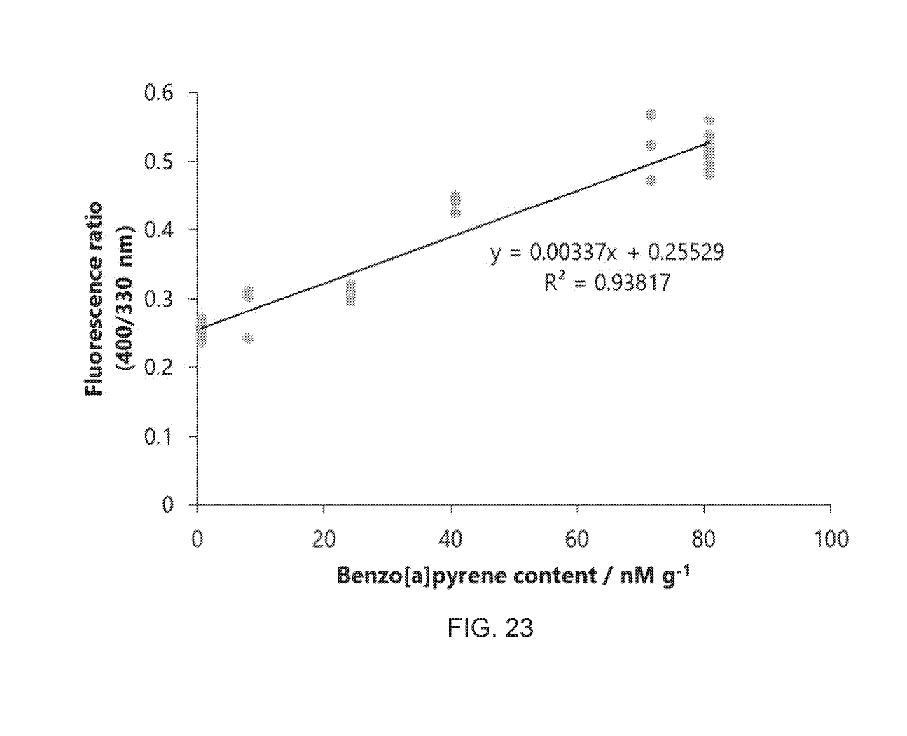

It was found in Example 1 that the 400/330 nm fluorescence ratio of BaP fluorescence at 255 nm excitation could be used to discriminate the lowest (sample 259) and highest (sample 263) BaP leaf content samples. The 400/330 nm fluorescence ratio has thus been calculated from the EEM data shown in FIGS. 18 to 20. The 400/330 nm fluorescence ratio is shown in FIG. 22 at varying concentrations of BaP. It can be seen from FIG. 22 that the 400/330 nm fluorescence ratio provides a relatively good linear relationship (R²=0.9052) with BaP content. The data produced in Example 1 for samples 259 and 263 washed in hexane was combined with these results to improve the linear fit, as is shown in FIG. 23. This additional data was acquired in duplicate across 30, 60, 180 and 300 s. None of the data were corrected for the weight of the tobacco leaf used to prepare each sample. The fluorescence ratio therefore seems to be independent of the exact weight of the tobacco leaf, the solvent sample volume, and the extraction time.

Determining the limit of detection as three times the standard deviation above the blank (in this case 11 repeats of 259 at extraction times 30-300 seconds) provides an estimated limit of detection of 8.6 nM/L.

It has thus been shown that the 400/330 nm fluorescence ratio at 255 nm excitation is linear with total BaP leaf content across six different leaf samples at extraction times 30-300 s without correction for the mass of tobacco.

### Determination of Ratio of BaP Extracted

The ratio of fluorescence response for BaP vs matrix co-extracts may be derived from Parallel Factor Analysis (PARAFAC) of the data resulting from fluorescence analysis of all the tobacco leaf samples identified above in Example 2.

The data in each of the EEMs constitute a 3-dimensional array of sample (or BaP concentration)×Excitation wavelength×Emission wavelength.

In order to construct a model that provides the required predictive power (or high correlation) between BaP concentration and fluorescence signal, PARAFAC was used to establish whether or not other variables, such as the signal from the co-extracted substances, contributed to the predictive power of the model. Another signal with a high correlation would perturb the model and confound its predictive capability.

If the fluorescence signal from BaP can be distinguished from the background based on visual peak-picking, or by examining the fluorescence at either a single emission wavelength or dual emission wavelengths, then this greatly simplifies the EEM data. However, if the fluorescence signal from BaP is too closely overlapping with the background fluorescence signal, more complex EEM modelling may be required. Probably the most common data analysis tool used for EEM deconvolution is PARAFAC.

An EEM matrix consists of a three-dimensional data array which can, therefore, often be decomposed into a unique solution. In PARAFAC, an alternating least-squares approach is taken to solve the three-way data matrix by minimising the sum of squares of the residuals in:

xᵢⱼₖ=Σ_{f=1}^{F}a_{if}b_{if}c_{kf}+eᵢⱼₖ

where xᵢⱼₖ is one element of the array, the fluorescence intensity of sample i at an emission wavelength j and an excitation wavelength k. F is the total number of components, and f is an individual component, where the component should ideally correspond to an individual fluorophore. The final term eᵢⱼₖ represents the residuals from unexplained signals (variation not accounted for by the modelling and noise). The model outputs the parameters a, b and c, which should represent the concentration, emission spectra and excitation spectra of the modelled fluorophores. The model assumes that the combination of individual fluorophores is a sum of the contribution from each fluorophore, in other words that the EEM of each component may vary in intensity but not wavelength.

The HORIBA Aqualog® was installed with a software package for PARAFAC Eigenvector's Solo, which has been used to distinguish BaP fluorescence in the presence of large and variable UV fluorescence background.

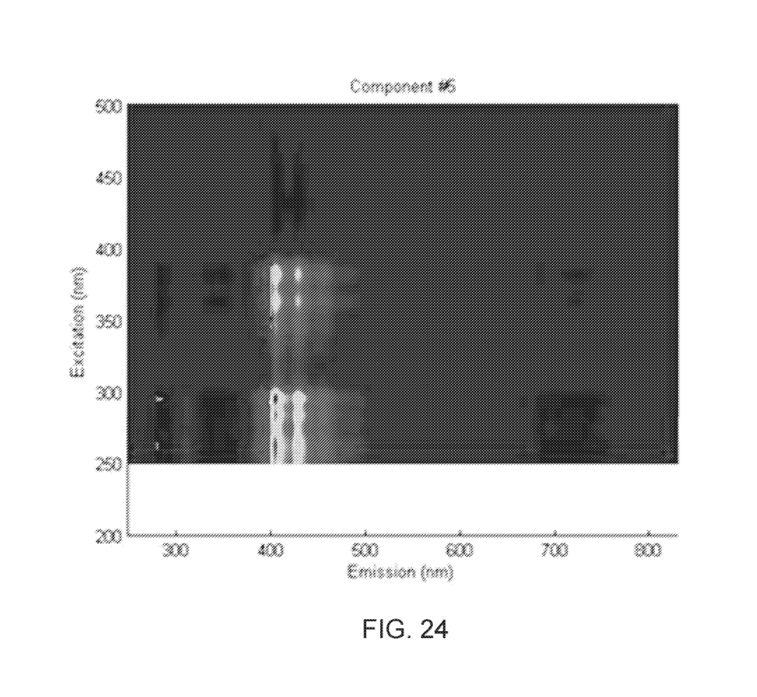

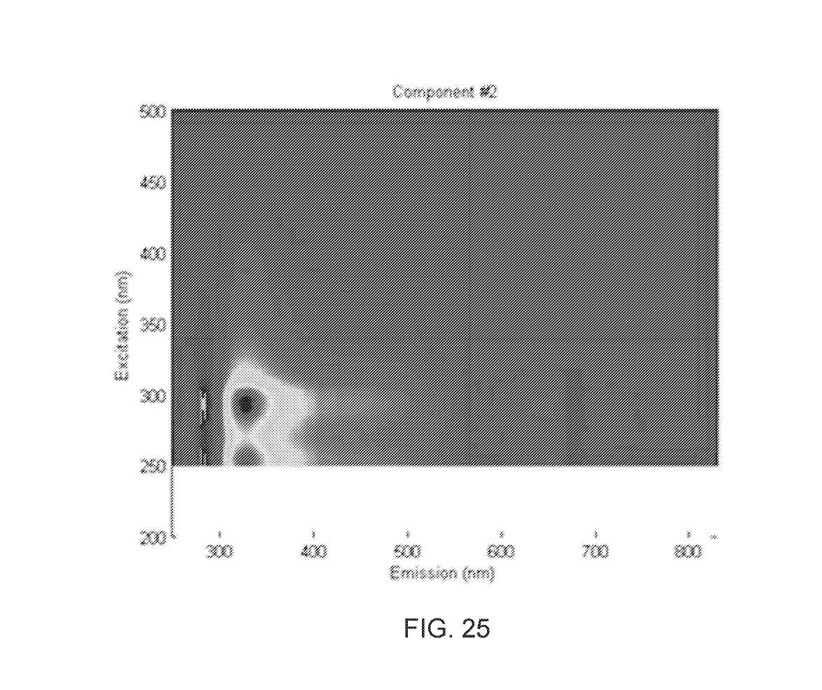

The ratio of matrix co-extracts in the hexane wash leaf samples identified above (having low, medium and high known BaP content) can be broadly extrapolated from FIGS. 24 to 26. FIG. 24 shows the fluorescence response for the BaP wavelengths of interest and, as it is a plot of the response across the full concentration range, comprises data points from all of the hexane leaf wash samples (from low to high BaP content); FIG. 25 shows the total background UV fluorescence. The slope ratio of the signal in FIG. 24 to that in FIG. 25 is 0.0441. As a percentage, this is 4.4%. This tells us that the ratio of BaP to fluorescent co-matrix extracts is approximately 4.4% of the BaP fluorescence signal.

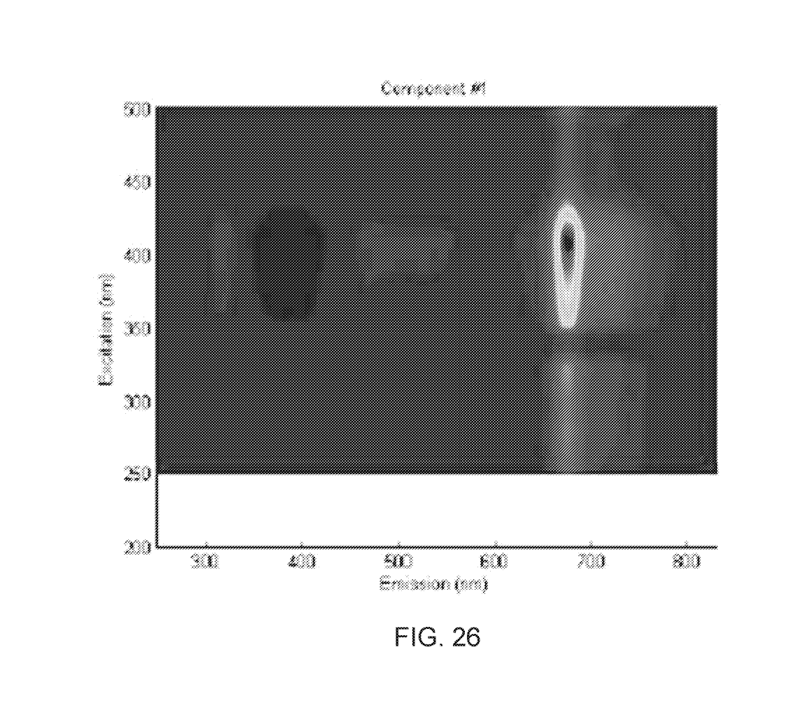

In order to further investigate the ratio of BaP to fluorescent matrix co-extracts, chlorophyll was selected as an example. Chlorophyll was selected because there is no contribution of chlorophyll to the fluorescence region of BaP, as the emission of chlorophyll is at 682 nm, so it is not interfering with the UV measurement. FIG. 26 shows the fluorescence response for chlorophyll.

The slope ratio of the signal in FIG. 24 (BaP) to that in FIG. 26 (chlorophyll) is 0.0394, which is 3.9%. This tells us that the ratio of BaP to chlorophyll extracted is approximately 3.9% of the BaP fluorescence signal currently. Of course, significantly higher levels of chlorophyll than BaP can be expected, but it can be potentially a suitable marker informing on how much over-extraction has occurred from the leaf.

### Example 3

18 blind samples with unknown levels of BaP were prepared from the leaf samples 258-263 identified in Example 2. Approximately 1 g of each blind tobacco leaf sample was placed in 25 mL hexane, and washed for a period of time of 180 s. The exact mass of each blind leaf sample is listed in Table 7 below.

TABLE 7
mass of each blind leaf sample

| Sample | Weight (g) |
|---|---|
| Blind 1 | 0.8382 |
| Blind 2 | 0.8716 |
| Blind 3 | 0.8446 |
| Blind 4 | 1.0264 |
| Blind 5 | 0.9146 |
| Blind 6 | 1.1194 |
| Blind 7 | 0.9845 |
| Blind 8 | 0.9239 |
| Blind 9 | 0.8917 |
| Blind 10 | 0.8605 |
| Blind 11 | 0.8364 |
| Blind 12 | 0.8313 |
| Blind 13 | 1.2352 |
| Blind 14 | 1.0691 |
| Blind 15 | 1.1479 |
| Blind 16 | 1.1925 |
| Blind 17 | 0.9736 |
| Blind 18 | 1.3370 |

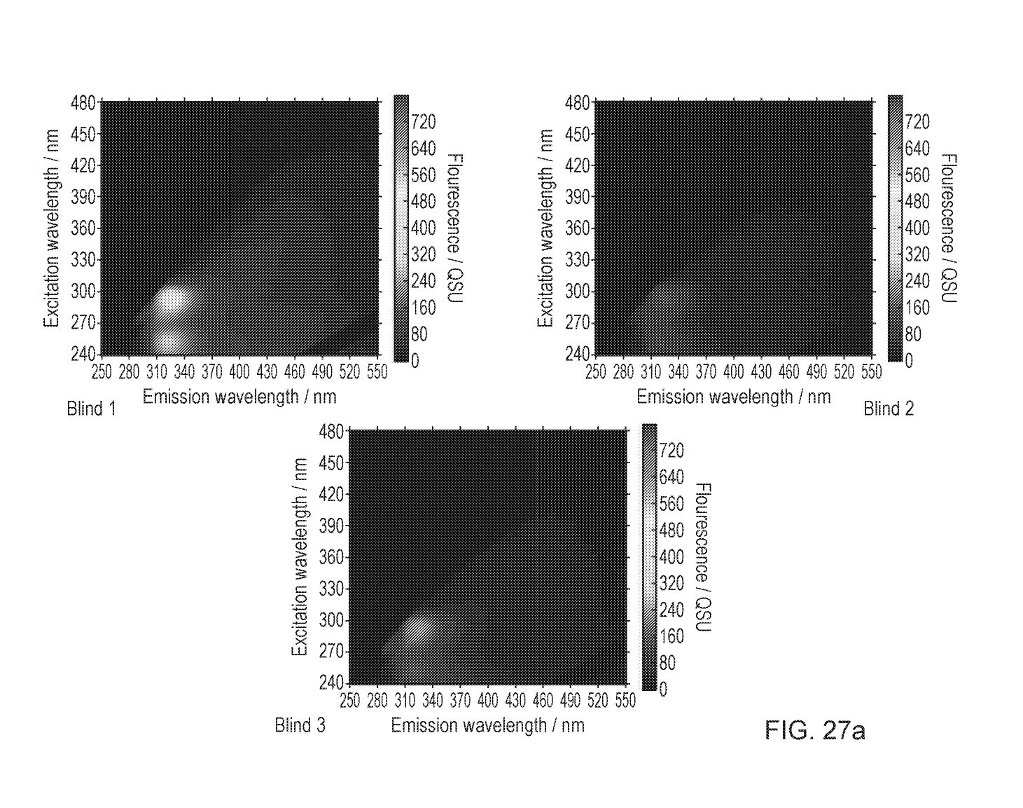

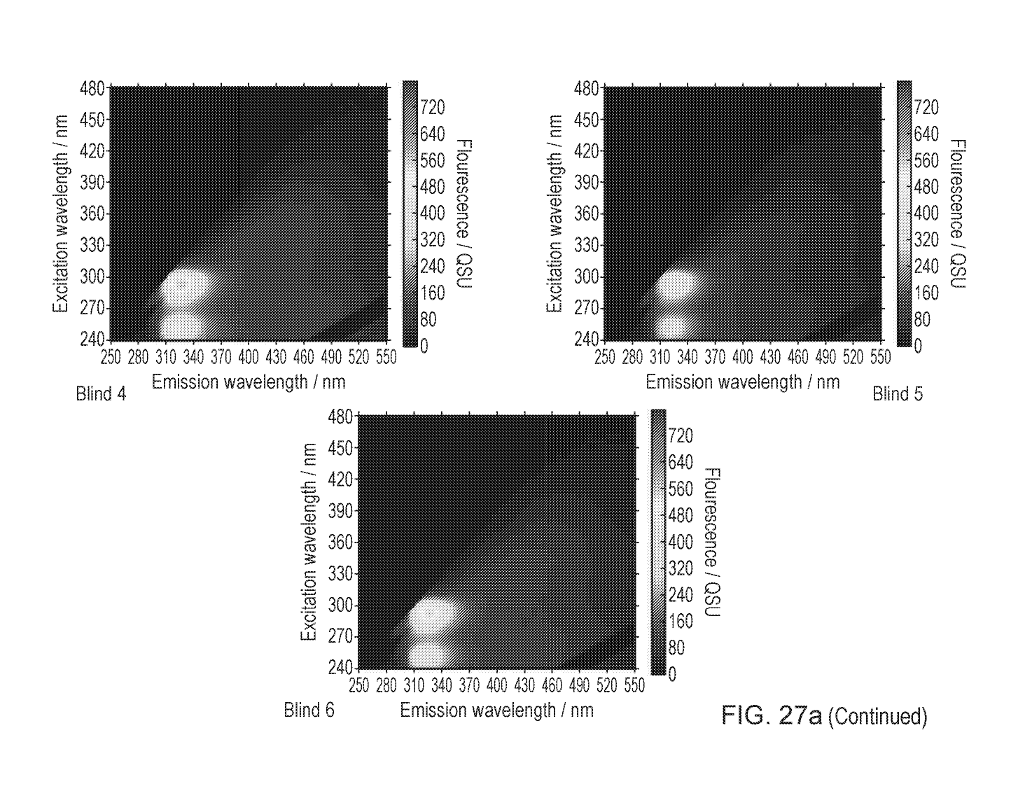

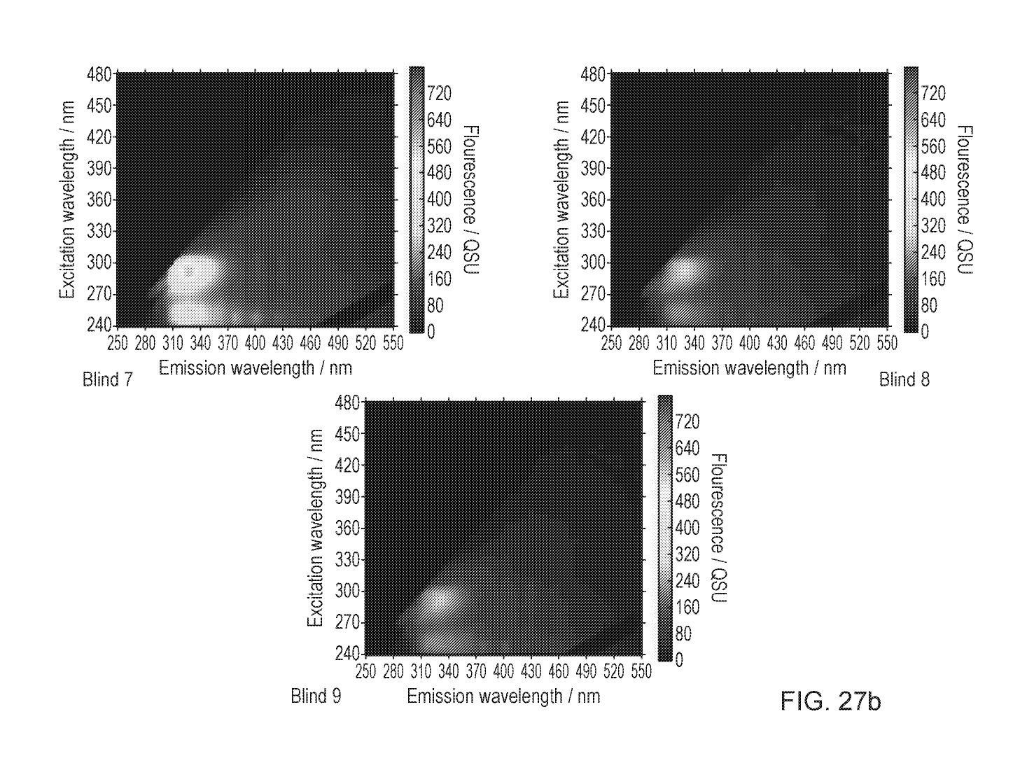

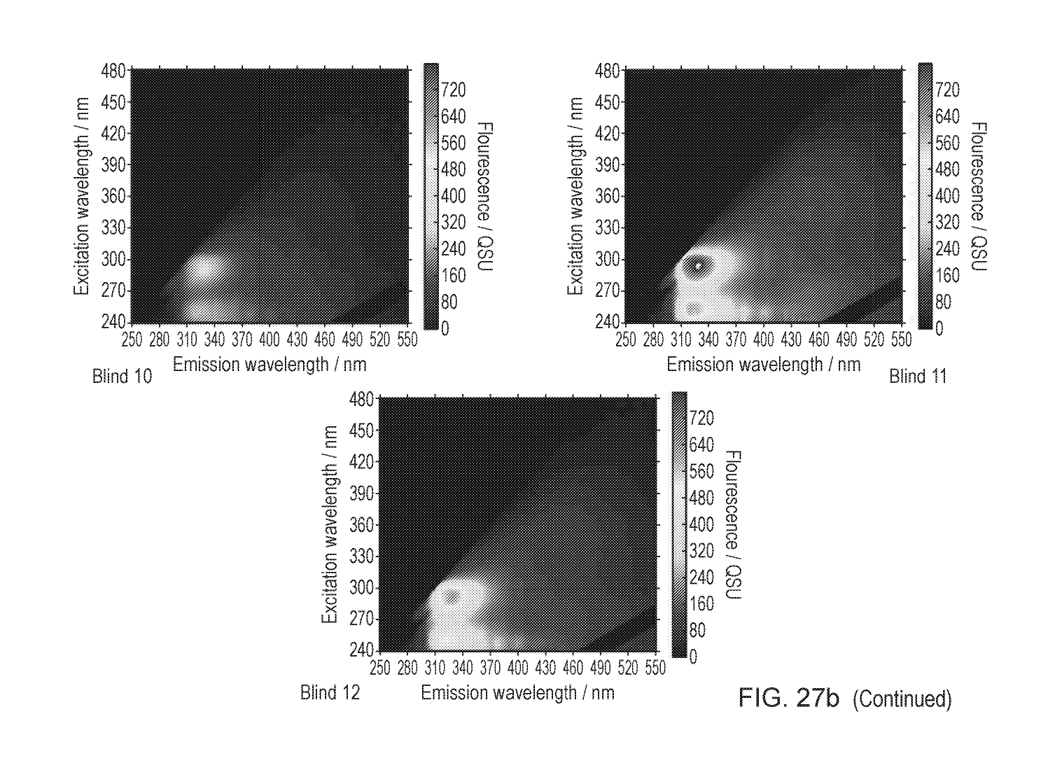

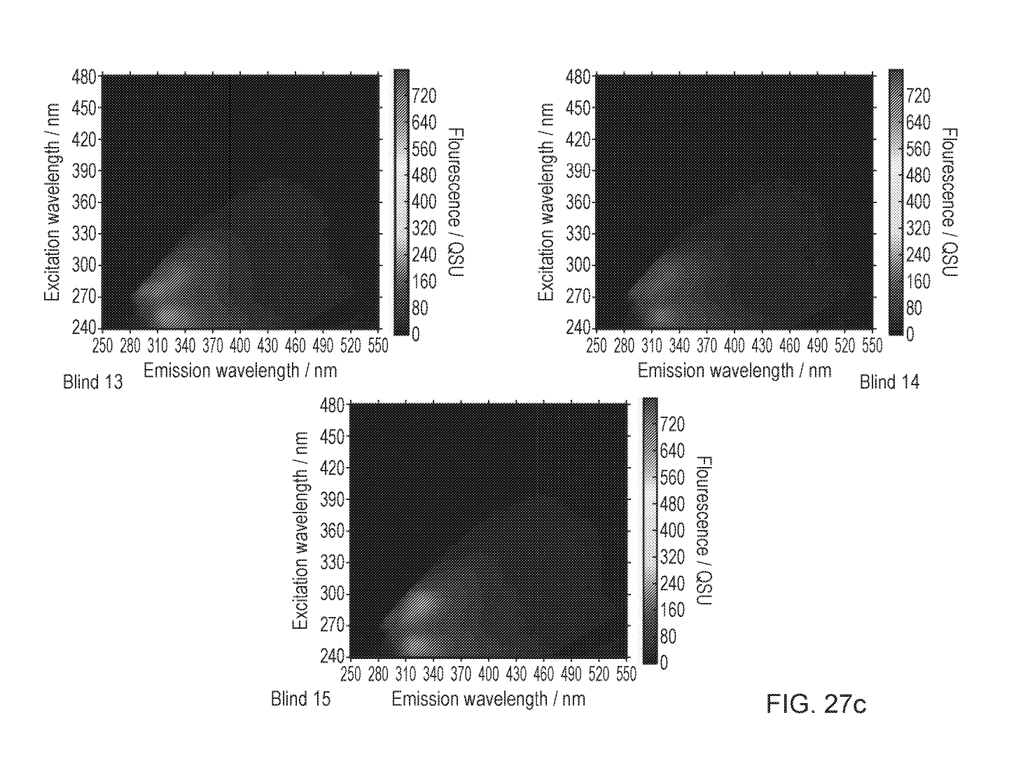

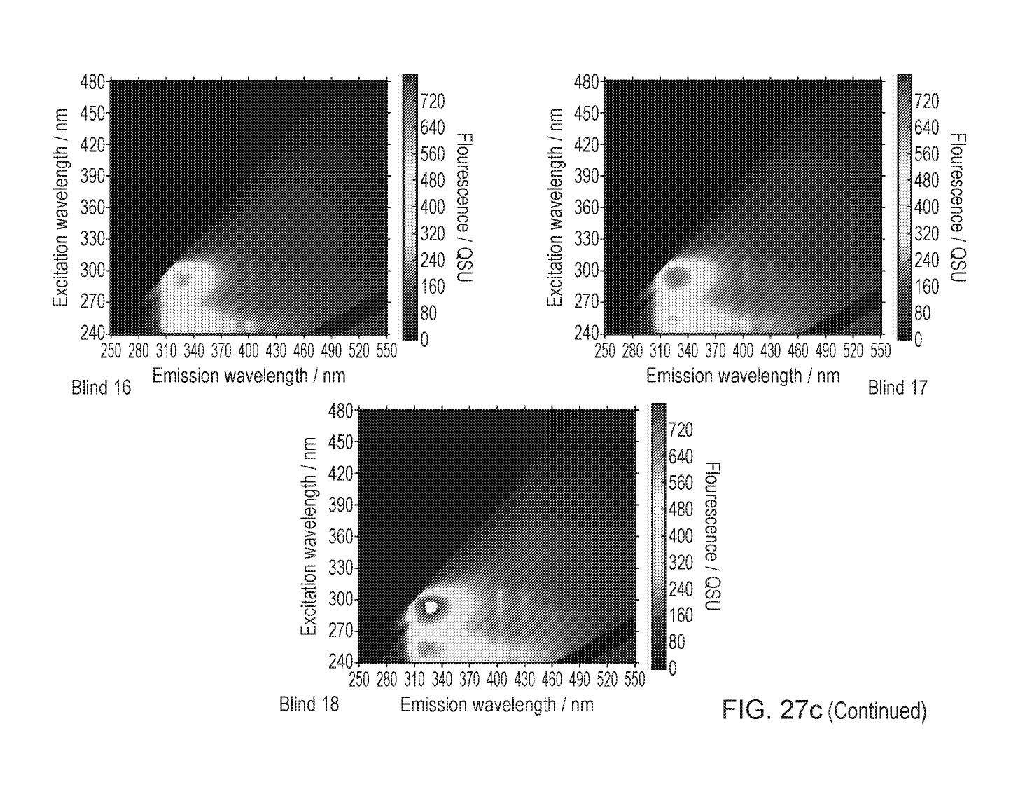

EEMs were acquired for each of the blind tobacco leaf samples using the HORIBA Aqualog® following the procedure described in Example 1. The EEMs for each of the 18 blind tobacco leaf samples are shown in FIGS. 27a-c.

A couple of independent methods were applied to establish the BaP level in each blind sample.

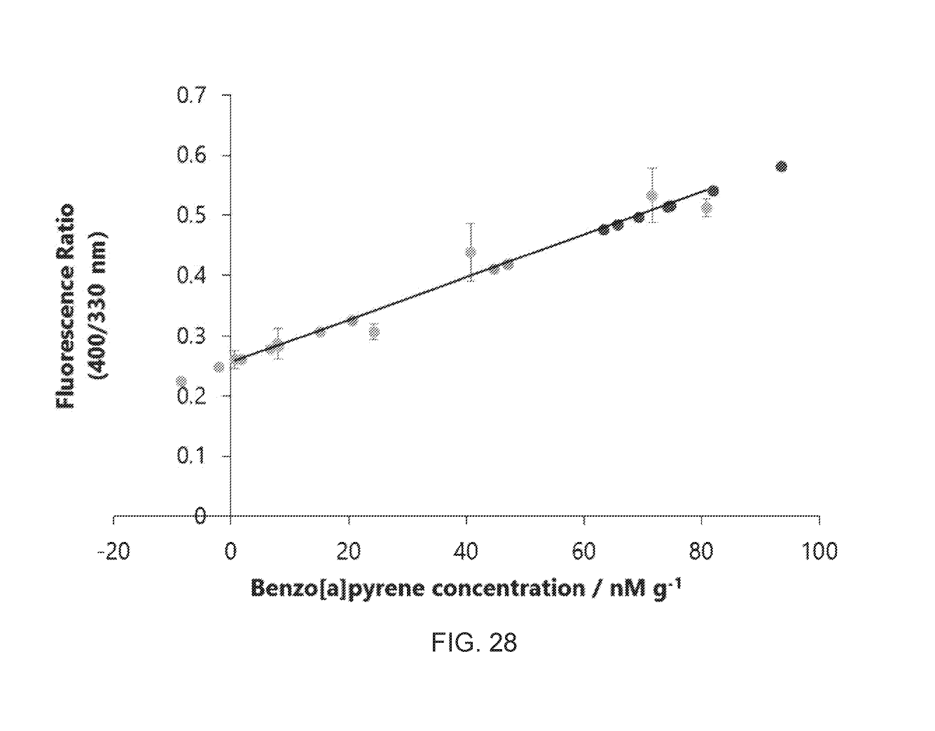

Firstly, using the calibration plot shown in FIG. 22, the 400/330 nm fluorescence ratio at 255 nm excitation for each blind sample was extracted from the individual EEMs and converted to predicted BaP concentration. The resultant plot is shown in FIG. 28, with low content BaP samples being defined as those with a concentration of <20 nM/L, medium content BaP samples defined as those with a concentration of from 20-60 nM/L, and high content BaP samples defined as those with a concentration of >60 nM/L. The data points shown with standard deviation error bars are for the average fluorescence ratio determined with the calibration samples of Example 2.

In addition, visual inspection of the EEMs in FIGS. 27a-c provided a good indication of the identity of each leaf wash sample from each blind sample, especially where the background to BaP fluorescence is particularly characteristic.

Using the above methods, an assignment was made for each of the blind samples, as shown in Table 8.

TABLE 8
assignment of each blind leaf sample to each leaf wash sample

| | | BaP content | | |
|---|---|---|---|---|
| | BaP content | assigned | | |
| | assigned using | using visual | | Actual |
| Blind sample | fluorescence | inspection of | Resulting | identity |
| number | ratio 400/330 nm | EEM | assignment | of sample |
| 1 | Low | Low | 258 | 258 |
| 2 | Low | Low | 258 | 258 |
| 3 | Low | Low | 258 | 258 |
| 4 | Low | Low | 259 | 259 |
| 5 | Low | Low | 259 | 259 |
| 6 | Low | Low | 259 | 259 |
| 7 | High | Medium | 261 | 260 |
| 8 | High | High | 260 | 260 |
| 9 | High | High | 260 | 260 |
| 10 | High | High | 260 | 261 |
| 11 | Medium | Medium | 261 | 261 |
| 12 | Medium | Medium | 261 | 261 |
| 13 | Low | Medium | 262 | 262 |
| 14 | Low | Medium | 262 | 262 |
| 15 | Medium | Medium | 262 | 262 |
| 16 | High | High | 263 | 263 |
| 17 | High | High | 263 | 263 |
| 18 | High | High | 263 | 263 |

Each of the blind samples was correctly assigned to the respective leaf wash sample 258-263, except for blind sample 7 and blind sample 10. In other words, 89% of the samples were assigned correctly. It is worth noting that the incorrect assignments of blind samples 7 and 10 are considered to be understandable since it requires differentiating between medium and high BaP content leaf wash samples.

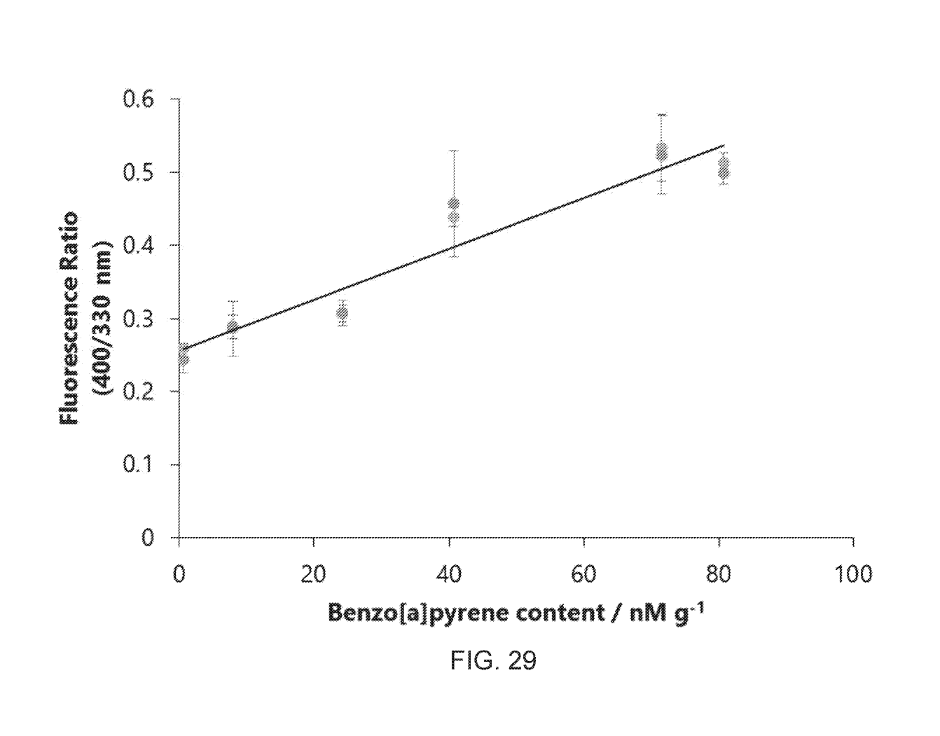

The fluorescence ratios of the blind samples, after correct assignment, were plotted with the fluorescence ratios of the calibration samples, as shown in FIG. 29. The excellent agreement between the calibration and the blind samples indicates that the fluorescent method can be used to identify the blind samples, and thus determine the level of BaP on the surface of tobacco leaves having an unknown level of BaP.

Determining the limit of detection as three times the standard deviation above the blank (in this case sample 259 across both calibration samples, data from Example 1 and blank samples) provides an estimated limit of detection of 10.8 nM. Good agreement was obtained between the limit of detection determined in this example, compared with that predicted from only two samples (259 and 263) analysed in Example 1, where a limit of detection of 10.0 nM was estimated. This indicates that the fluorescence ratio 400/330 nm is a marker of BaP content across six different leaf types at extraction times 30-300 seconds, without correction for the exact mass of tobacco.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of appropriate combinations of the disclosed elements, components, features, parts, steps, means, etc., other than those specifically described herein. In addition, this disclosure may include other inventions not presently claimed, but which may be claimed in the future.

1. A method for determining the level of a polycyclic compound of interest present on the surface of a tobacco leaf, the method comprising the steps of:
(a) washing the surface of the tobacco leaf with a solvent, the solvent comprising at least a non-polar solvent, such that no greater than about 10 wt. % of the polycyclic compound of interest present on the surface of the tobacco leaf is removed from the surface of the tobacco leaf;
(b) collecting the solvent from step (a); and
(c) subjecting the collected solvent to fluorescence spectroscopy to determine the level of the polycyclic compound of interest.

2. A method according to claim 1 wherein the polycyclic compound of interest is an aromatic polycyclic compound.

3. A method according to claim 2 wherein the polycyclic compound of interest is a pyrene.

4. A method according to claim 3 wherein the pyrene is a benzopyrene.

5. A method according to claim 4 wherein the benzopyrene is benzo(alpha)pyrene.

6. A method according to any one of claims 1 to 5 wherein the solvent is hexane.

7. A method according to any one of claims 1 to 6 wherein from about 0.01 to about 10 wt. % of the polycyclic compound of interest present on the surface of the tobacco leaf is removed from the surface of the tobacco leaf.

8. A method according to any one of claims 1 to 7 wherein from about 2 to about 8 wt. % of the polycyclic compound of interest present on the surface of the tobacco leaf is removed from the surface of the tobacco leaf.

9. A method according to any one of claims 1 to 8 wherein from about 4 to about 6 wt. % of the polycyclic compound of interest present on the surface of the tobacco leaf is removed from the surface of the tobacco leaf

10. A method according to any one of claims 1 to 9 wherein the collected solvent subjected to fluorescence spectroscopy contains the polycyclic compound of interest in an amount of about 5 pg/mL to about 500 pg/mL.

11. A method according to any one of claims 1 to 10 wherein the collected solvent subjected to fluorescence spectroscopy contains the polycyclic compound of interest in an amount of about 5 ng per g of tobacco leaf to about 500 ng per g of tobacco leaf.

12. A method according to any one of claims 1 to 11 wherein the amount of solvent used to wash the tobacco leaf is from about 10 to about 50 ml of solvent per 1 g of tobacco leaf.

13. A method according to any one of claims 1 to 12 wherein the amount of solvent used to wash the tobacco leaf is from about 20 to about 40 ml of solvent per 1 g of tobacco leaf.

14. A method according to any one of claims 1 to 13 wherein in step (b) the tobacco leaf is washed with solvent for a period of less than about 10 minutes.

15. A method according to any one of claims 1 to 14 wherein in step (b) the tobacco leaf is washed with solvent for a period of from about 0.5 to about 5 minutes.

16. A method according to any one of claims 1 to 15 wherein in step (b) the tobacco leaf is washed at a temperature of about 10 to about 30° C.

17. A method according to any one of claims 1 to 16 wherein in step (b) the tobacco leaf is washed at atmospheric pressure.

18. A method according to any one of claims 1 to 17 wherein the fluorescence spectroscopy comprises identifying at least a first peak and a second peak in the spectrum of the collected solvent, the first peak corresponding to a first specific emission wavelength of the polycyclic compound of interest and the second peak corresponding to a second specific emission wavelength of the polycyclic compound of interest.

19. A method according to claim 18 wherein the first peak is emission with a wavelength in the range 310 to 370 nm.

20. A method according to claim 18 or 19 wherein the second peak is emission with a wavelength in the range 385 to 425 nm.

21. A method according to claim 18, 19 or 20 comprising the step of comparing the intensities of the first peak and the second peak relative to each other so as to obtain a fluorescence ratio.

22. A method according to claim 21 comprising the step of comparing the fluorescence ratio to pre-determined fluorescence ratios in order to determine the level of the polycyclic compound of interest on the surface of the tobacco leaf.

23. A device for determining the level of a polycyclic compound of interest present in a solvent, the device comprising:
(a) a receptacle for the solvent containing the polycyclic compound of interest;
(b) an excitation source for initiating fluorescence of the polycyclic compound of interest;
(c) a sensor for determining fluorescence; and
(d) a processor operable to
(i) identify at least a first peak and a second peak in the emission spectrum of the collected solvent, the first peak corresponding to a first specific emission wavelength for the polycyclic compound of interest and the second peak corresponding to a second specific emission wavelength of the polycyclic compound of interest;
(ii) compare the intensities of the two peaks relative to each other so as to obtain an fluorescence ratio; and
(iii) compare the fluorescence ratio to pre-determined fluorescence ratios in order to determine the level of the polycyclic compound of interest.

24. A method as substantially defined herein with reference to the Examples.

25. A device as substantially defined herein with reference to the Examples.
